(19) United States
(12) Patent Application Publication
(10) Pub. No.: US 2012/0021924 A1
(43) Pub. Date: Jan. 26, 2012

(54) DETECTION AND MODULATION OF CYTOCHROME C ACETYLATION

(75) Inventors: Angela Valeska Hafner, Boston, MA (US); David A. Sinclair, Chestnut Hill, MA (US)

(73) Assignee: President and Fellows of Harvard College, Cambridge, MA (US)

(21) Appl. No.: 13/125,405

(22) PCT Filed: Oct. 23, 2009
(86) PCT No.: PCT/US09/05778

§ 371 Date: Oct. 5, 2011

Related U.S. Application Data
(60) Provisional application No. 61/107,841, filed on Oct. 23, 2008.

(51) Int. Cl. C40B 30/00; G01N 33/566; H01J 49/26; C12Q 1/34

(52) U.S. Cl. 506/7; 435/18; 435/7.1; 250/282

(57) ABSTRACT

The invention relates to detection and modulation of cytochrome c acetylation. The invention has diagnostic and therapeutic applications for neurodegenerative disorders and cancer.

### RELATED APPLICATION

This application claims the benefit under 35 U.S.C. §119(e) of U.S. Provisional Application Ser. No. 61/107,841, entitled “Detection and Modulation of Cytochrome C Acetylation,” filed on Oct. 23, 2008, which is herein incorporated by reference in its entirety.

### GOVERNMENT INTEREST

This invention was made with government support under AG027916, awarded by the National Institutes of Health. The government has certain rights in the invention.

### FIELD OF THE INVENTION

The invention pertains to methods of diagnosis and treatment of neurodegenerative diseases and cancer.

### BACKGROUND OF THE INVENTION

Cytochrome c is a haem-containing protein within the inner mitochondrial membrane, wherein it is a component of the electron transport chain. Cytochrome c is also part of the intrinsic apoptotic pathway. In cells that are undergoing apoptosis, cytochrome c is released from the mitochondrial membrane to interact with apoptotic protease-activating factor-1 (APAF1), forming the apoptosome, which activates caspase proteases involved in mediating cell death.

Release of cytochrome c and commitment of a cell to apoptosis is a highly regulated process, and represents a target regulatory step for disorders associated with apoptosis.

Cytochrome c activity has been shown to be regulated through multiple factors including BCL2 family members, caspases, heat-shock proteins, proteins that affect fission/fusion of the mitochondria, calcium levels, regulation of the redox states of cytochrome c, nitrosylation, histone H1.2 and cytosolic p53 (Ow et al., (2008) Nat Rev Mol Cell Biol 9:532-542).

### SUMMARY OF THE INVENTION

Described herein is a novel approach for regulating cytochrome c, involving modulation of acetylation. Cytochrome c acetylation is revealed to be associated with cells undergoing apoptosis. Thus, detection of cytochrome c acetylation levels has applications for diagnosis of neurodegenerative disorders, and deacetylation of cytochrome c represents a therapeutic approach for treatment of neurodegenerative disorders. Furthermore, induction of apoptosis through acetylation of cytochrome c, and monitoring of cytochrome c levels, have diagnostic and therapeutic applications for cancer.

Aspects of the invention relate to methods for characterizing a subject's risk of a neurodegenerative disorder by detecting the level of acetylated cytochrome c in a sample from the subject, wherein an elevated level of acetylated cytochrome c in the sample from the subject, relative to a predetermined value, is indicative of an increased risk of a neurodegenerative disorder. The level of acetylated cytochrome c in a sample can be detected by any means known to one of ordinary skill in the art such as using an antibody that specifically binds acetylated cytochrome c, and/or through the use of mass spectrometry.

In some embodiments of the invention, methods for characterizing a subject's risk of a neurodegenerative disorder include detecting acetylation of a lysine residue corresponding to residue K40 in a full-length, wild-type cytochrome c polypeptide, in a sample from the subject, wherein the presence of acetylation of a lysine residue corresponding to residue K40 in a full-length, wild-type cytochrome c polypeptide in a sample from the subject indicates that the subject has an increased risk of a neurodegenerative disorder. In certain embodiments acetylation of lysine residue K40 is detected by mass spectrometry.

In some embodiments of the invention, methods for characterizing a subject's risk of a neurodegenerative disorder include detecting acetylation of a lysine residue corresponding to residue K74 in a full-length, wild-type cytochrome c polypeptide, in a sample from the subject, wherein the presence of acetylation of a lysine residue corresponding to residue K74 in a full-length, wild-type cytochrome c polypeptide in a sample from the subject indicates that the subject has an increased risk of a neurodegenerative disorder. In certain embodiments, acetylation of lysine residue K74 is detected by mass spectrometry. In some embodiments methods for characterizing a subject's risk of a neurodegenerative disorder include detecting acetylation of lysine residues corresponding to residues K40 and K74 in a full-length, wild-type cytochrome c polypeptide, in a sample from the subject, wherein the presence of acetylation of lysine residues corresponding to residues K40 and K74 in a full-length, wild-type cytochrome c polypeptide indicates that the subject has an increased risk of neurodegenerative disorder. In certain embodiments acetylation of lysine residues K40 and K74 is detected by mass spectrometry.

Aspects of the invention relate to methods for diagnosing a neurodegenerative disorder in a subject, including detecting the level of acetylated cytochrome c in a sample from the subject, wherein an elevated level of acetylated cytochrome c in the sample from the subject, relative to a predetermined value, is indicative of a neurodegenerative disorder. In certain embodiments the level of acetylated cytochrome c in a sample from the subject is detected by using an antibody that specifically binds acetylated cytochrome c and/or by using mass spectrometry.

Further aspects of the invention relate to methods for evaluating the efficacy of a therapy in a subject with a neurodegenerative disorder, including detecting the level of acetylation of cytochrome c in a sample from the subject, wherein the level of acetylation of cytochrome c in the sample from the subject, relative to a predetermined value, is indicative of whether the therapy is efficacious. In certain embodiments the level of acetylated cytochrome c in a sample from the subject is detected by using an antibody that specifically binds acetylated cytochrome c and/or by using mass spectrometry.

Also described herein are methods for treating a subject having a neurodegenerative disorder, including administering an effective amount of a compound to a subject in need of such a treatment to decrease the level of acetylated cytochrome c in the subject below a predetermined value, wherein the compound is a compound that activates a sirtuin. In some embodiments, the invention involves inhibiting apoptosis in a cell in which cytochrome c is acetylated by contacting the cell with an agent that deacetylates cytochrome c. The agent that deacetylates cytochrome c can be a deacetylase protein such as a sirtuin. In some embodiments the sirtuin is SIRT3.

Further aspects of the invention relate to methods for determining whether a cancer patient should be treated with an agent that acetylates cytochrome c by performing an assay to determine whether a patient has a cancer that exhibits deacetylation of lysine residues corresponding to residues K40 and K74 in a full-length, wild-type cytochrome c polypeptide, wherein the patient is a candidate for treatment with a composition that acetylates cytochrome c if the patient has a cancer that exhibits deacetylation of lysine residues corresponding to residues K40 and K74 in a full-length, wild-type cytochrome c polypeptide.

Described herein are methods for inducing apoptosis in a cell in which cytochrome c is deacetylated by contacting the cell with an agent that acetylates cytochrome c, to thereby induce apoptosis in the cell. In some embodiments the cell is in vivo and the method further comprises contacting the cell with an additional therapeutic agent. Some embodiments of the invention involve methods for decreasing viability of a cancer cell that exhibits deacetylation of cytochrome c by contacting the cancer cell that exhibits deacetylation of cytochrome c with an agent that acetylates cytochrome c in an amount effective to decrease the viability of the cancer cell. In some embodiments the cell is in vivo and the method further comprises contacting the cell with an additional therapeutic agent.

Also described herein are isolated antibodies or antigen-binding fragments thereof that bind specifically to an epitope of acetylated cytochrome c polypeptide, wherein the epitope comprises an acetylated residue that corresponds to residue K40 in a full-length, wild-type, human cytochrome c amino acid sequence. In some embodiments the isolated antibody or antigen-binding fragment thereof specifically binds to the epitope with a binding affinity of about 1×10⁻⁶M, 1×10⁻⁷ M, 1×10⁻⁸ M, 1×10⁻⁹M, 1×10⁻¹° M, 5×10⁻¹° M, or 1×10⁻¹¹ M or less. In certain embodiments the antibody or antigen-binding fragment thereof is attached to a detectable label. Also included herein are nucleic acid molecules encoding such antibodies, hybridomas containing these nucleic acid molecules, and hybridoma cell lines producing such antibodies. Aspects of the invention also involve expression vectors including an isolated nucleic acid molecule encoding the antibodies or antigen-binding fragments described herein, host cells transformed by or transfected with such expression vectors, and plasmids that produce the antibodies or antigen-binding fragments described herein. In some embodiments, the invention relates to compositions comprising the antibodies or antigen-binding fragments described herein.

Also described herein are isolated antibodies or antigen-binding fragments thereof that bind specifically to an epitope of acetylated cytochrome c polypeptide, wherein the epitope comprises an acetylated residue that corresponds to residue K74 in a full-length, wild-type, human cytochrome c amino acid sequence. In some embodiments the isolated antibody or antigen-binding fragment thereof specifically binds to the epitope with a binding affinity of about 1×10⁻⁶M, 1×10⁻⁷ M, 1×10⁻⁸ M, 1×10⁻⁹M, 1×10⁻¹⁰ M, 5×10⁻¹⁰ M, or 1×10⁻¹¹ M or less. In certain embodiments the antibody or antigen-binding fragment thereof is attached to a detectable label. Also included herein are nucleic acid molecules encoding such antibodies, hybridomas containing these nucleic acid molecules, and hybridoma cell lines producing such antibodies. Aspects of the invention also involve expression vectors including an isolated nucleic acid molecule encoding the antibodies or antigen-binding fragments described herein, host cells transformed by or transfected with such expression vectors, and plasmids that produce the antibodies or antigen-binding fragments described herein. In some embodiments, the invention relates to compositions comprising the antibodies or antigen-binding fragments described herein.

Also described herein are methods for identifying compounds that modulate the deactylase activity of SIRT3. Such methods include contacting an acetylated cytochrome c polypeptide substrate and a SIRT3 deacetylase in the presence of a test compound, and determining the level of acetylation of the cytochrome c polypeptide substrate in the presence of the test compound. In some embodiments, the cytochrome c polypeptide substrate comprises at least one acetylated lysine residue corresponding to residues K40 and/or K74 of full-length, wild-type human cytochrome c polypolypeptide. A decrease in the level of acetylation of the cytochrome c polypeptide substrate in the presence of the test compound as compared to a control is indicative of a compound that increases SIRT3 deactylase activity. An increase in the level of acetylation of the cytochrome c polypeptide substrate in the presence of the test compound as compared to a control is indicative of a compound that decreases SIRT3 deactylase activity.

In some embodiments, the level of acetylation of the cytochrome c polypeptide substrate pool is determined using mass spectrometry. In some embodiments, the mass spectrometry is electrospray ionization (ESI) mass spectrometry or matrix-assisted laser desorption/ionization (MALDI) mass spectrometry.

In some embodiments of the foregoing methods, the cytochrome c polypeptide substrate comprises a single polypeptide species, while in other embodiments the cytochrome c polypeptide substrate comprises a mixture of two or more polypeptides species.

In some embodiments, the cytochrome c polypeptide substrate comprises a full-length cytochrome c polypeptide. In other embodiments, the cytochrome c polypeptide substrate is a fragment of cytochrome c comprising at least one lysine residue corresponding to residues K40 and/or K74 of full-length, wild-type human cytochrome c polypeptide. In still other embodiments, the cytochrome c polypeptide substrate is a fusion of a fragment of cytochrome c comprising at least one lysine residue corresponding to residues K40 and/or K74 of full-length, wild-type human cytochrome c polypeptide.

In some embodiments, the test compound is a small molecule, such as small organic molecules. In some embodiments, the test compound is a library of molecules, which library may, in certain embodiment, include small molecules, such as small organic molecules.

In some embodiments, the SIRT3 deacetylase is from a cell or tissue lysate.

In some embodiments, the cytochrome c polypeptide substrate is in a cell.

In some embodiments, the SIRT3 is a catalytically active fragment of full-length (human) SIRT3 capable of deacetylating a cytochrome c substrate comprising acetylated K40 and/or K74 in the presence of NAD⁺ or a NAD⁺ analog.

Also described herein are acetylated polypeptide substrates for use in determining the activity of SIRT3. The substrates include a fragment of cytochrome c comprising at least one acetylated lysine residue corresponding to residues K40 and/or K74 of full-length, wild-type human cytochrome c polypeptide. In some embodiments, the polypeptide substrate is a fusion of a fragment of cytochrome c comprising at least one acetylated lysine residue corresponding to residues K40 and/or K74 of full-length, wild-type human cytochrome c polypeptide. Also provided herein are kits including the foregoing acetylated polypeptide substrates.

These and other aspects of the invention, as well as various embodiments thereof, will become more apparent in reference to the drawings and detailed description of the invention.

### BRIEF DESCRIPTION OF THE DRAWINGS

The accompanying drawings are not intended to be drawn to scale. For purposes of clarity, not every component may be labeled in every drawing. In the drawings:

FIG. 1 presents a table demonstrating the results of mass spectrometry analysis, identifying sites of acetylation in cytochrome c. The protein sequence of mouse cytochrome C indicated in FIG. 1 is provided as SEQ ID NO:1.

FIG. 2 presents a table demonstrating the results of mass spectrometry analysis, identifying sites of acetylation in cytochrome c, in the absence of transfected hSIRT3.

FIG. 3 presents a table demonstrating the results of mass spectrometry analysis in the presence of transfected hSIRT3, showing that cytochrome c is deacetylated in the presence of SIRT3.

FIG. 4 presents a sequence alignment of cytochrome c protein in a variety of species. The protein sequences of human, mouse, Drosophila and S. cerevisiae cytochrome C proteins are provided as SEQ ID NO:2, SEQ ID NO:1, SEQ ID NO:3 and SEQ ID NO:4 respectively.

FIG. 5 presents a Western blot showing that acetylation of K74 can be detected using the anti-PAN antibody (Cell Signaling Technology, Beverly, Mass.).

FIG. 6 presents a Western blot showing the results of an immunoprecipitation experiment demonstrating that cytochrome c interacts with SIRT3.

FIG. 7 presents a Western blot showing the results of a deacetylation assay demonstrating that SIRT3 can deacetylate endogenous cytochrome c.

FIG. 8 presents a schematic demonstrating experimental procedures for behavioral experiments conducted with SIRT3 knockout mice.

FIG. 9 presents a graph and a schematic indicating the effects of kainic acid on primary cerebellar granule neurons in wild-type and SIRT3 knockout mice.

FIG. 10 presents graphs indicating weight change in wild-type and SIRT3 knockout mice.

FIG. 11 presents a graph demonstrating learning time in female wild-type and SIRT3 knockout mice.

FIG. 12 presents a graph demonstrating learning time in male wild-type and SIRT3 knockout mice.

FIG. 13 presents a schematic demonstrating experimental procedures for fear conditioning experiments.

FIG. 14 presents a graph demonstrating loss of hippocampus and amygdala neurons caused by kainic acid injections.

FIG. 15 presents a graph showing the results of contextual fear conditioning experiments in SIRT3 knockout mice.

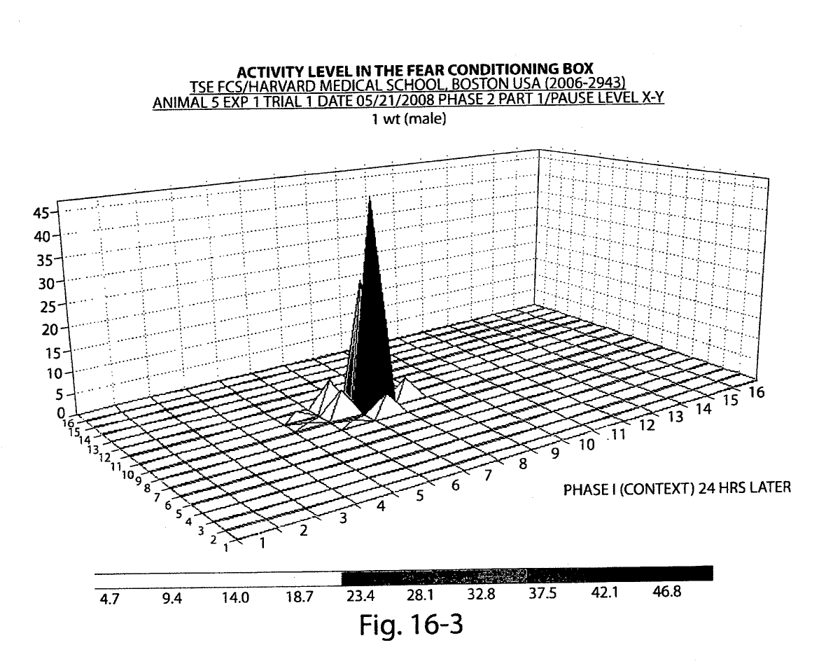

FIG. 16 presents graphs indicating activity levels in fear conditioning experiments.

FIG. 17 presents a schematic depicting the experimental procedure followed for testing locomotor activity in mice using an Open Field Test.

FIG. 18 presents a graph indicating weights of wild-type and SIRT3 knockout mice that were tested in the Open Field Test and in the Water Maze test.

FIG. 19 presents a graph indicating distance traveled in the Open Field Test by wild-type and SIRT3 knockout mice.

FIG. 20 presents a graph indicating the speed of movement in the Open Field Test by wild-type and SIRT3 knockout mice.

FIG. 21 presents a graph indicating results from day 1 of the water maze experiment using a visible platform marked with a flag, conducted with wild-type and SIRT3 knockout mice.

FIG. 22 presents schematics indicating results of Probe Trial 2 of the water maze experiments on wild-type mice.

FIG. 23 presents schematics indicating results of Probe Trial 2 of the water maze experiments on SIRT3 knockout mice.

FIG. 24 presents a graph indicating the time spent in different quadrants by wild-type and SIRT3 knockout mice in Probe Trial 1 of the water maze experiments.

FIG. 25 presents a graph indicating the percentage of time spent in different quadrants by wild-type and SIRT3 knockout mice in Probe Trial 1 of the water maze experiments.

FIG. 26 presents a graph indicating the time spent in different quadrants by wild-type and SIRT3 knockout mice in Probe Trial 2 of water maze experiments.

FIG. 27 presents a graph indicating the percentage of time spent in different quadrants by wild-type and SIRT3 knockout mice in Probe Trial 2 of water maze experiments.

FIG. 28 presents an image of a gel showing the results of immunoprecipitation experiments from hippocampal lysates of wild-type and SIRT3 knockout mice using the PAN antibody (acetylated-lysine antibody, Cell Signaling Technology, Beverly, Mass.) and revealing hyperacetylation of proteins in the hippocampus of SIRT3 knockout mice.

FIG. 29 presents a schematic of a kit associated with the invention. The kit (10) shown in FIG. 29 includes a set of containers for housing a compound or compounds (12) or (14) such as a compound for activating SIRT3. The kit optionally contains instructions (20). Additional components may also be included in the kit.

### DETAILED DESCRIPTION OF THE INVENTION

The invention is based at least in part on the surprising discovery that cytochrome c is acetylated on at least two lysine (K) residues, K40 and K74. Deacetylation of cytochrome c is revealed to be mediated through interaction with the deacetylase SIRT3, which is shown herein to be involved in protecting neurons from cell death. Monitoring and regulating acetylation of cytochrome c provides a diagnostic and therapeutic resource for a variety of diseases, including diseases associated with cell death.

Aspects of the invention relate to the discovery of acetylation of the cytochrome c polypeptide. As used herein, the terms “protein” and “polypeptide” are used interchangeably and thus the term polypeptide may be used to refer to a full-length polypeptide and may also be used to refer to a fragment of a full-length polypeptide. The term, “acetylated cytochrome c polypeptide” means a cytochrome c polypeptide that is acetylated at one or more lysine residues. In some embodiments, more than one lysine (K) residue is acetylated. In some embodiments, only one lysine residue is acetylated. In some embodiments, a cytochrome c polypeptide may be acetylated only at the residue that corresponds to the K40 residue of wild-type, full-length human cytochrome c polypeptide. In some embodiments, a cytochrome c polypeptide may be acetylated only at the residue that corresponds to the K74 residue of wild-type, full-length human cytochrome c polypeptide. In some embodiments, a cytochrome c polypeptide may be acetylated on residues that correspond to residue K40 and residue K74 of wild-type, full-length human cytochrome c polypeptide. In some embodiments, a cytochrome c polypeptide may be acetylated on residues that correspond to residues K40 and K74 of wild-type, full-length human cytochrome c polypeptide, and on one or more other lysine residues. In some embodiments, K40 and/or K74 or other lysine positions that are acetylated may be used in methods and/or products of the invention.

The residue in position 40 of wild-type, full-length human cytochrome c polypeptide is a lysine, and this lysine in the wild-type, full-length human polypeptide and the residue that corresponds to this position in fragments and in mutated forms of cytochrome c may be referred to herein as “K40”. Cytochrome c in which the K40 residue is acetylated may be referred to herein as “K40-acetylated cytochrome c”.

The residue in position 74 of wild-type, full-length human cytochrome c polypeptide is a lysine, and this lysine in the wild-type, full-length polypeptide and the residue that corresponds to this position in fragments and in mutated forms of cytochrome c may be referred to herein as “K74”. Cytochrome c in which the K74 residue is acetylated may be referred to herein as “K74-acetylated cytochrome c”.

A wild-type, full-length human cytochrome c polypeptide has the amino acid sequence set forth as Genbank Accession No. NP_{—}061820. An acetylated wild-type, full-length human cytochrome c polypeptide also has the amino acid sequence set forth in Genbank Accession No. NP_{—}061820, but is acetylated at one or more of its lysine residues. A nucleic acid sequence encoding human wild-type, full-length cytochrome c is set forth as Genbank Accession No. NM_{—}018947. The nucleic acid and protein sequences of mouse cytochrome c correspond to Genbank Accession Nos. X01756 and CAA25899 respectively.

There may be allelic variation in cytochrome c polypeptide sequences of the invention including wild-type cytochrome c polypeptide sequences and/or mutant cytochrome c polypeptide sequences. As used herein, the term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides with altered amino acid sequences. An allelic variant of a polypeptide is a polypeptide encoded by an allelic variant of a gene. It will be understood by those of ordinary skill in the art that such allelic variations may occur in full-length wild-type and mutant cytochrome c polypeptides and in fragments of wild-type and mutant polypeptides. Cytochrome c polypeptides of the invention may be allelic variants of wild-type cytochrome c or mutant cytochrome c polypeptide sequences. One of ordinary skill in the art will be able to identify which residues of variants of wild-type and mutant cytochrome c polypeptide correspond to residues of wild-type cytochrome c polypeptide using routine methods.

### Fragments

In some embodiments, the acetylated lysine residue in a fragment of cytochrome c polypeptide is referred to as an acetylated K40 residue or K74 residue even though the fragment is not a full-length cytochrome c polypeptide. Those of ordinary skill in the art can readily determine the correspondence of an acetylated residue in a cytochrome c polypeptide sequence (wild-type or mutant) with a residue in a full-length, wild-type cytochrome c polypeptide using routine sequence comparison methods.

In some aspects, the invention may include the synthesis of acetylated full-length cytochrome c polypeptides or acetylated fragments thereof. Synthesis methods of the invention may include any art-known synthetic methods such as the acetylation of an existing natural or synthetic cytochrome c polypeptide, or the incorporation of an acetylated lysine residue in a cytochrome c polypeptide during synthesis. Incorporation of acetylated lysine may include the following acetylation step, which occurs at the epsilon-amino groups of lysines:

Lysine+acetyl-CoA->Acetyl-Lysine+H₂O

As used herein with respect to polypeptides, proteins, or fragments thereof, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may be, but need not be, substantially pure. The term “substantially pure” means that the proteins or polypeptides are essentially free of other substances with which they may be found in production, nature, or in vivo systems to an extent practical and appropriate for their intended use. Substantially pure polypeptides may be obtained naturally or produced using methods described herein and may be purified with techniques well known in the art. Because an isolated protein may be admixed with therapeutic components in a preparation, such as a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

According to some aspects of the invention, fragments of full-length, wild-type or mutant cytochrome c polypeptides are provided. Fragments of the invention are preferably fragments that retain a distinct functional capability of the polypeptide. Functional capabilities which can be retained in a fragment include acetylation, interaction with antibodies, and interaction with other polypeptides or fragments thereof. Polypeptide fragments can be synthesized using art-known methods, and tested for function using the methods exemplified herein.

A fragment of an acetylated cytochrome c polypeptide may comprise at least 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 102, or more (including each integer in between) contiguous amino acids of cytochrome c polypeptide having a consecutive sequence found in wild-type human cytochrome c polypeptide or a modified cytochrome c polypeptide sequence as described herein. In some embodiments, a fragment includes a lysine residue that corresponds to K40 and/or K74 of full-length, wild-type human cytochrome c polypeptide. Residues that correspond to K40 and K74 may or may not be acetylated. Fragments of acetylated cytochrome c polypeptides can be prepared using synthetic methods known in the art or may be natural fragments of acetylated cytochrome c polypeptides. Such fragments are useful for a variety of purposes, including in the preparation of molecules that bind specifically to synthetic and naturally acetylated cytochrome c polypeptides and in immunoassays well known to those of ordinary skill in the art, including competitive binding immunoassays. In some embodiments, fragments of acetylated cytochrome c could be used to assay SIRT3 activity or to inhibit SIRT3 activity.

One of ordinary skill in the art will understand how to prepare fragments of full-length wild-type or mutant cytochrome c polypeptide. An acetylated fragment of a full-length wild-type or mutant cytochrome c polypeptide may include an acetylated lysine that corresponds to the K40 and/or K74 lysine of wild-type, full-length human cytochrome c polypeptide and/or may include an acetylated lysine that corresponds to a different lysine of wild-type, full-length human cytochrome c polypeptide. Also, in some embodiments of the invention, a fragment of cytochrome c polypeptide may include a K40 and/or K74 residue and one or more additional lysine residues, and one, each, some, or none of the lysines may be acetylated.

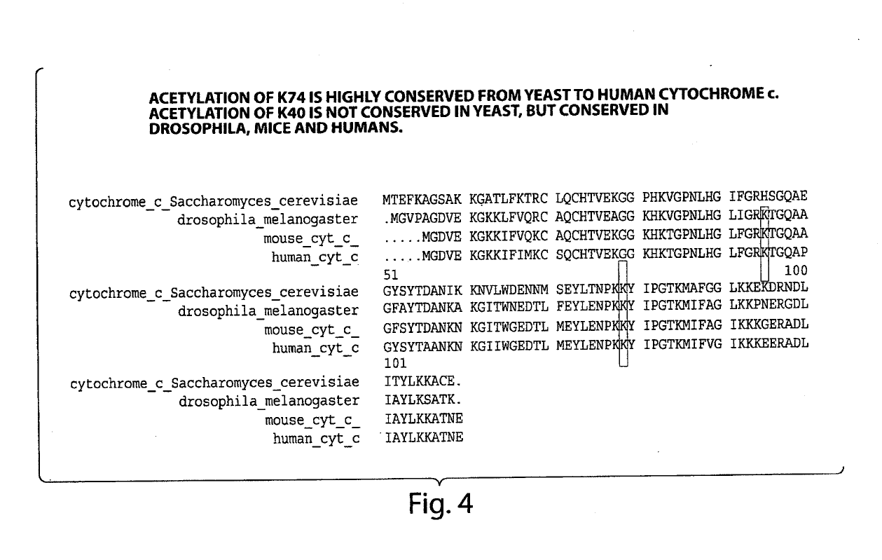

One of ordinary skill in the art is aware that functional homologs of human cytochrome c exist in multiple species. Acetylated polypeptides including full-length proteins and fragments of full-length proteins from other species, that are functionally homologous to human cytochrome c are compatible with the instant invention. One of ordinary skill in the art is aware of techniques to identify a residue in a homologous protein that is functionally homologous to residues K40 or K74 in human cytochrome c. For example, FIG. 4 presents a sequence alignment of cytochrome c proteins in a variety of species. While human cytochrome c K74 is conserved in each species, in some species this residue is not in position 74 of the cytochrome c protein in that species. However based on sequence alignment and other methods known to those of ordinary skill in the art, it would be apparent which residue in the cytochrome c polypeptide from a given species is functionally homologous to residue K74 in human cytochrome c.

It should be appreciated that aspects of the invention encompass detection of acetylation of the human wild-type full-length cytochrome c protein, and also detection of acetylation of the cytochrome c protein in fragments, variants and mutants of the human cytochrome c protein. Furthermore, aspects of the invention encompass detection of acetylation of the cytochrome c protein from any other species, including detection of acetylation of the wild-type full-length cytochrome c protein from any other species, and detection of acetylation of fragments, variants and mutants of the cytochrome c protein from any other species.

A “modified” wild-type or mutant cytochrome c polypeptide or fragment thereof may include deletions, point mutations, truncations, amino acid substitutions and/or additions of amino acids or non-amino acid moieties. Modifications of a polypeptide of the invention may be made by modification of the nucleic acid that encodes the polypeptide or alternatively, modifications may be made directly to the polypeptide, such as by cleavage, addition of a linker molecule, addition of a detectable moiety, such as biotin, addition of a carrier molecule, and the like. Modifications also embrace fusion proteins comprising all or part of the polypeptide's amino acid sequence.

In general, modified cytochrome c polypeptides include polypeptides that are modified specifically to alter a feature of the polypeptide unrelated to its physiological activity. For example, cysteine residues can be substituted or deleted to prevent unwanted disulfide linkages. Polypeptide modifications can be made by selecting an amino acid substitution, deletion, and/or addition, and a modified polypeptide may be synthesized using art-known methods. Modified polypeptides then can be tested for one or more activities (e.g., antibody binding, antigenicity, etc.) to determine which modification provides a modified polypeptide with the desired properties.

The skilled artisan will also realize that conservative amino acid substitutions may be made in a polypeptide to provide functionally equivalent polypeptides, i.e., modified cytochrome c polypeptides that retain a functional capability of a wild-type or mutant cytochrome c polypeptide. As used herein, a “conservative amino acid substitution” refers to an amino acid substitution that does not alter the relative charge or size characteristics of the protein in which the amino acid substitution is made. Modified cytochrome c polypeptides can be prepared according to methods for altering polypeptide sequence and known to one of ordinary skill in the art such. Exemplary functionally equivalent cytochrome c polypeptides include conservative amino acid substitutions of a cytochrome c polypeptide, or fragments thereof. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in a cytochrome c polypeptide typically are made by alteration of a nucleic acid encoding the polypeptide. Such substitutions can be made by a variety of methods known to one of ordinary skill in the art. For example, amino acid substitutions may be made by PCR-directed mutation, site-directed mutagenesis, or by chemical synthesis of a gene encoding the cytochrome c polypeptide. Where amino acid substitutions are made to a small fragment of a polypeptide, the substitutions can be made by directly synthesizing the polypeptide. The activity of functionally equivalent fragments of cytochrome c polypeptides can be tested by cloning the gene encoding the altered polypeptide into a bacterial or mammalian expression vector, introducing the vector into an appropriate host cell, expressing the altered polypeptide, and testing for a functional capability of the polypeptide as disclosed herein.

As described above, a fragment of a full-length wild-type or mutant cytochrome c polypeptide may be a synthetic polypeptide. As used herein, the term “synthetic” means artificially prepared. A synthetic polypeptide is a polypeptide that is synthesized and is not a naturally produced polypeptide molecule (e.g., not produced in an animal or organism). It will be understood that the sequence of a natural polypeptide (e.g., an endogenous polypeptide) may be identical to the sequence of a synthetic polypeptide, but the latter will have been prepared using at least one synthetic step.

As used herein, a synthetic acetylated polypeptide is a polypeptide acetylated with a synthetic method, which may be, but is not limited to a method of the invention. An acetylated polypeptide of the invention may be a naturally acetylated polypeptide (e.g., an endogenous acetylated polypeptide) or may be a synthetic acetylated polypeptide. Although a synthetic acetylated polypeptide may differ from a natural acetylated polypeptide, an antibody raised against a synthetic polypeptide of the invention will specifically bind with high affinity the synthetic polypeptide epitope against which it was raised, and will also specifically bind with high affinity the natural epitope in a polypeptide. Thus, even though an acetylated epitope of a synthetic polypeptide may differ slightly in amino acid sequence from the same epitope in a natural acetylated polypeptide, an antibody raised against a synthetic acetylated epitope of the invention specifically binds, in most cases, with high affinity to the natural acetylated epitope and to a synthetic acetylated epitope. Antibodies of the invention generated using a synthetic acetylated polypeptide specifically bind, in most cases, with high affinity to natural and synthetic acetylated polypeptides and are able to distinguish between natural (heterogeneous) acetylated and natural non-acetylated polypeptides and also to distinguish between synthetic acetylated and synthetic non-acetylated polypeptides.

### Cytochrome c Deacetylation by SIRT3

Histone deacetylase proteins (HDACs) constitute four different classes. Class III HDACs, which are NAD⁺-dependent deacetylases, are known as sirtuins. Sirtuins are conserved proteins that deacetylate both histone and non-histone cellular targets. In humans, seven sirtuins have been identified (SIRT1-7), with individual sirtuin proteins exhibiting distinct subcellular localizations and functions. SIRT3 protein has been reported to exhibit both nuclear and mitochondrial localizations, and SIRT3 function has been associated with metabolism and longevity.

In the Examples section, it is demonstrated that SIRT3 binds to and deacetylates cytochrome c. It is also demonstrated that mice in which SIRT3 function has been knocked out exhibit decreased cell survival, indicating a function for SIRT3 in neuroprotection. Furthermore, it is shown herein that SIRT3 plays a role in memory formation and fear conditioning.

Aspects of the invention relate to regulating acetylation and deacetylation of cytochrome c. In some embodiments, methods of the invention involve increasing the activity or protein level of a sirtuin such as SIRT3 in order to decrease the acetylation of cytochrome c. In some embodiments the activity or protein level of a sirtuin such as SIRT3 is increased through administering the sirtuin gene or protein. In some embodiments the activity or protein level of a sirtuin such as SIRT3 is increased through administering a compound that increases the protein level or increases the activity a sirtuin. Methods for activating sirtuins, and non-limiting examples of compounds for activating sirtuins are provided by formulas 1-25, 30, and 32-65 in US Patent Publication 2006/0025337, incorporated by reference herein in its entirety. Methods and compounds for modulating sirtuins are also presented in US Patent Publications: 2007/0043050, 2007/0037865, 2007/0037827, 2007/0037809, 2007/0014833, 2006/0276416, 2006/0276393 and 2006/0229265, and in U.S. Pat. No. 7,345,178, all of which are incorporated herein by reference in their entirety.

The invention further encompasses screening methods for identifying compounds that modulate sirtuins such as SIRT3. An compound that modulates the activity of a sirtuin such as SIRT3 may in some embodiments be a nucleic acid (e.g., an aptamer), a polypeptide, or a small molecule, e.g., a small organic molecule. Non-limiting examples of compounds for modulating sirtuin activity are provided in US Patent Publication 2006/0025337, incorporated by reference herein in its entirety, such as the molecules of formulas 1-25, 30, and 32-65, or analogs thereof. It should be appreciated that a wide variety of compounds and/or compound libraries are appropriate for screening methods described herein.

Assays may be conducted in a cell-based or cell-free format. For example, an assay may comprise incubating (or contacting) a sirtuin, such as SIRT3, and a test compound under conditions in which a sirtuin can be activated by an compound known to activate the sirtuin, and monitoring or determining the level of activation of the sirtuin in the presence of the test compound relative to the absence of the test compound. The level of activation of a sirtuin can be determined by determining its ability to deacetylate a substrate. Exemplary substrates are acetylated polypeptides, or libraries or pools of polypeptides. In some embodiments, the substrate is a cytochrome c polypeptide. In some embodiments, a substrate contains a single polypeptide species, while in other embodiments, it contains a mixture of two or more polypeptides species. In some embodiments, the substrate comprises one or more cytochrome c polypeptides that have one or more acetylated residues. In certain embodiments, the substrate comprises one or more cytochrome c polypeptides that have an acetylated lysine residue corresponding to residues K40 and/or K74. Polypeptide substrates can include, for example, full-length proteins and/or protein fragments and/or heterologous fusions, alone or in combination. Polypeptides can be of varying lengths. In some embodiments, a cytochrome c polypeptide substrate comprises a fusion of a fragment of cytochrome c comprising at least one lysine residue corresponding to residues K40 and/or K74. A fusion of a fragment of cytochrome c can encompass any fragment of a cytochrome c polypeptide fused to a fragment of any other polypeptide. In some embodiments, the cytochrome c polypeptide substrate is in a cell. Substrates used in screening assays may in some embodiments be fluorogenic.

It should be appreciated that methods and compositions described herein can encompass a full-length SIRT3 protein, or a portion thereof. In some embodiments, a biologically active portion of SIRT3 may be used in accordance with the methods described herein. A biologically active portion of SIRT3 refers to a portion of the protein having a biological activity, such as the ability to deacetylate an acetylated substrate, such as cytochrome c, or a fragment of cytochrome c comprising at least one lysine residue corresponding to residues K40 and/or K74, in the presence of nicotinamide adenine dinucleotide (NAD⁺) or an NAD⁺ analog. Biologically active portions of SIRT3 can in some embodiments encompass the NAD+ binding domain and/or the substrate binding domain. In other embodiments, a biologically active portion of SIRT3 may be a fragment of a SIRT3 protein that is produced by cleavage with a mitochondrial matrix processing peptidase (MPP) and/or a mitochondrial intermediate peptidase (MIP). The SIRT3 deacetylase used in methods described herein can be from a cell or tissue lysate. The assays described herein can be used to determine if a portion of SIRT3 is a biologically active portion of SIRT3.

In some embodiments, the reaction may be conducted for about 30 minutes and stopped, e.g., with nicotinamide. Assays similar to those described in the HDAC fluorescent activity assay/drug discovery kit (AK-500, BIOMOL Research Laboratories) may be used to determine the level of acetylation. Similar assays are described in Bitterman et al. (2002) J. Biol. Chem. 277:45099. The level of activation of the sirtuin in an assay may be compared to the level of activation of the sirtuin in the presence of one or more (separately or simultaneously) compounds, which may serve as positive or negative controls. Sirtuins for use in the assays may be full length SIRT3 proteins or biologically active portions thereof. In some embodiments, proteins for use in the assays include N-terminal portions of SIRT3. Methods for screening for compounds that modulate sirtuins such as SIRT3 are incorporated by reference from U.S. Pat. No. 7,544,497, and US Patent Publications 2009/0221020, 2008/0293081 and 2006/0252076.

Methods may comprise (i) contacting a cell comprising a sirtuin such as SIRT3 with a cytochrome c polypeptide substrate under conditions appropriate for the sirtuin to deacetylate the polypeptide and (ii) determining the level of acetylation of the polypeptide, wherein a different level of acetylation of the polypeptide in the presence of the test compound relative to a control (such as the absence of the test compound) indicates that the test compound modulates the activity of the sirtuin in vivo. It should be appreciated that other substrates besides cytochrome c would also be compatible in such assays for identifying compounds that modulate the activity of the sirtuin.

In one embodiment, a screening assay comprises (i) contacting the sirtuin such as SIRT3 with a test compound and an acetylated substrate under conditions appropriate for the sirtuin to deacetylate the substrate in the absence of the test compound; and (ii) determining the level of acetylation of the substrate, wherein a lower level of acetylation of the substrate in the presence of the test compound relative to the absence of the test compound indicates that the test compound stimulates deacetylation by the sirtuin, whereas a higher level of acetylation of the substrate in the presence of the test compound relative to the absence of the test compound indicates that the test compound inhibits deacetylation by the sirtuin.

Methods for identifying an compound that modulates, e.g., stimulates or inhibits, a sirtuin such as SIRT3 in vivo may comprise (i) contacting a cell with a test compound and a substrate that is capable of entering a cell in the presence of an inhibitor of class I and class II HDACs under conditions appropriate for the sirtuin to deacetylate the substrate in the absence of the test compound; and (ii) determining the level of acetylation of the substrate, wherein a lower level of acetylation of the substrate in the presence of the test compound relative to the absence of the test compound indicates that the test compound stimulates deacetylation by the sirtuin, whereas a higher level of acetylation of the substrate in the presence of the test compound relative to the absence of the test compound indicates that the test compound inhibits deacetylation by the sirtuin. A preferred substrate is an acetylated polypeptide, which may also be fluorogenic. The method may further comprise lysing the cells to determine the level of acetylation of the substrate. In some embodiments, substrates may be added to cells at a concentration ranging from about 1 μM to about 10 mM, preferably from about 10 μM to 1 mM, even more preferably from about 100 μM to 1 mM, such as about 200 μM.

In some embodiments, methods for identifying a compound that activates a sirtuin such as SIRT3 may involve mass spectrometry, discussed further below. Methods of using mass spectrometry for identifying compounds that modulate the activity of deacetylase proteins are incorporated by reference from US Patent Publication 2009/0221020. Mass spectrometry can be used to identify the level of acetylation of cytochrome c or any other substrate of a sirtuin such as SIRT3. In some embodiments, a method for identifying a compound that activates a deacetylase includes contacting a cytochrome c polypeptide with a sirtuin such as SIRT3, or a biologically active portion thereof, in the presence of a test compound, wherein the cytochrome c polypeptide comprises at least one acetylated lysine residue, and determining the level of acetylation of the cytochrome c polypeptide using mass spectrometry, wherein a decrease in the level of acetylation of the polypeptide in the presence of the test compound as compared to a control is indicative of a compound that activates a deacetylase. Mass spectrometry can in some embodiments encompass electrospray ionization (ESI) mass spectrometry and/or matrix-assisted laser desorption/ionization (MALDI) mass spectrometry.

In some embodiments, methods for determining the activity of a deacetylase such as a sirtuin comprise: contacting a polypeptide with a cell or tissue lysate comprising a deacetylase, wherein the polypeptide comprises at least one acetylated lysine residue; and determining the level of acetylation of the polypeptide using mass spectrometry, wherein a decrease in the level of acetylation of the polypeptide is indicative of deacetylase activity. The deacetylase can be SIRT3 and can be in a cell or tissue lysate. The cytochrome c polypeptide can be in a cell.

In some embodiments, the concentration of a polypeptide substrate is below the Kₘ of the sirtuin, such as SIRT3, for the polypeptide substrate. For example, the concentration of the polypeptide substrate can be at least 2, 3, 4, 5, 6, 7, 8, 9, 10, 11, 12, 13, 14, 15 or more than 15 fold below the Kₘ of the sirtuin for the polypeptide substrate.

A compound subjected to or identified by screening methods described herein may for example be a small molecule, such as a small organic molecule. Such small molecules are well known in the art; examples of such molecules are provided herein and in publications such as U.S. Pat. No. 7,544,497 and US 2009/0221020. Aspects of the invention encompass preparing a quantity of such a compound, or analog thereof, and in some embodiments conducting therapeutic profiling of the compound, or analog thereof, for efficacy and toxicity in animals. Methods for conducting therapeutic profiling are familiar to one of ordinary skill in the art. In some aspects, methods involve formulating the compound in pharmaceutical formulations, using standard methods. The invention encompasses manufacturing of pharmaceutical preparations containing compounds described herein, or compounds identified using methods described herein, or analogs thereof, having a suitable animal toxicity profile. Pharmaceutical preparations containing compounds described herein, or compounds identified using methods described herein, or analogs thereof, having a suitable animal toxicity profile can be marketed to healthcare providers. Methods for preparing quantities of a compound or analog thereof, conducting therapeutic profiling of the compound or analog thereof, formulating a compound in pharmaceutical formulations, and manufacturing a pharmaceutical preparation containing a compound, are incorporated by reference from U.S. Pat. No. 7,544,497 and US Patent Publication 2009/0221020.

Aspects of the invention also relate to acetylated polypeptide substrates for use in determining the activity of SIRT3, comprising a fragment of cytochrome c comprising at least one acetylated lysine residue corresponding to residues K40 and/or K74. The polypeptide substrate can be a fusion of a fragment of cytochrome c comprising at least one acetylated lysine residue corresponding to residues K40 and/or K74. Such polypeptides can be chemically synthesized, produced recombinantly, or by any other methods routinely employed in the art. The polypeptides can be acetylated according to standard methods routinely practiced in the art. Aspects of the invention also relate to kits comprising such acetylated polypeptide substrates, which can be used in the screening methods described herein.

Neurodegenerative Disorders

Aspects of the invention relate to diagnosis and treatment of disorders. As used herein, “disorder” refers to any pathological condition associated with elevated or reduced cytochrome c acetylation. In some embodiments a disorder or condition associated with elevated acetylated cytochrome c is a neurodegenerative disorder. As used herein, the term “neurodegenerative disorder” refers to disorders, diseases or conditions that are caused by the deterioration of cell and tissue components of the nervous system.

Some non-limiting examples of neurodegenerative disorders include stroke, Alzheimer's disease, Parkinson's disease, Huntington's disease, Periventricular leukomalacia (PVL), amyotrophic lateral sclerosis (ALS, “Lou Gehrig's disease”), ALS-Parkinson's-Dementia complex of Guam, Friedrich's Ataxia, Wilson's disease, multiple sclerosis, cerebral palsy, progressive supranuclear palsy (Steel-Richardson syndrome), bulbar and pseudobulbar palsy, diabetic retinopathy, multi-infarct dementia, macular degeneration, Pick's disease, diffuse Lewy body disease, prion diseases such as Creutzfeldt-Jakob, Gerstmann-Straussler-Scheinker disease, Kuru and fatal familial insomnia, primary lateral sclerosis, degenerative ataxias, Machado-Joseph disease/spinocerebellar ataxia type 3 and olivopontocerebellar degenerations, spinal and spinobulbar muscular atrophy (Kennedy's disease), familial spastic paraplegia, Wohlfart-Kugelberg-Welander disease, Tay-Sach's disease, multisystem degeneration (Shy-Drager syndrome), Gilles De La Tourette's disease, familial dysautonomia (Riley-Day syndrome), Kugelberg-Welander disease, subacute sclerosing panencephalitis, Werdnig-Hoffmann disease, synucleinopathies (including multiple system atrophy), Sandhoff disease, cortical basal degeneration, spastic paraparesis, primary progressive aphasia, progressive multifocal leukoencephalopathy, striatonigral degeneration, familial spastic disease, chronic epileptic conditions associated with neurodegeneration, Binswanger's disease, and dementia (including all underlying etiologies of dementia).

### Cancer

Aspects of the invention also relate to diagnosis and treatment of cancer. As used herein, the term “cancer” refers to an uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject.

As used herein, the term “cancer” includes, but is not limited to, the following types of cancer: breast cancer (including carcinoma in situ), biliary tract cancer; bladder cancer; brain cancer including glioblastomas and medulloblastomas; cervical cancer; choriocarcinoma; colon cancer; endometrial cancer; esophageal cancer; gastric cancer; hematological neoplasms including acute lymphocytic and myelogenous leukemia; T-cell acute lymphoblastic leukemia/lymphoma; hairy cell leukemia; chromic myelogenous leukemia, multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; mesothelioma, neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Merkel cell carcinoma, Kaposi's sarcoma, basal cell carcinoma, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Non-limiting examples of precancerous conditions include dysplasia, premalignant lesions, adenomatous colon polyp, and carcinoma in-situ such as Ductal carcinoma in-situ (DCIS), etc. Other cancers that can be treated with methods of the invention will be known to those of ordinary skill in the art. In some embodiments of the invention, the cancer is melanoma. In certain embodiments the cancer is adenocarcinoma. In some embodiments the cancer is a solid tumor cancer. A cancer that may be treated or assayed using methods of the invention also may include breast cancer, lung cancer, prostate cancer, mesothelioma, etc.

### Measuring Acetylation of Cytochrome c

The invention, in some aspects, includes various assays to determine levels of acetylated cytochrome c polypeptide, and to detect acetylation of cytochrome c on specific residues (e.g., K40 and/or K74). Methods of the invention that are useful to determine levels of acetylated cytochrome c polypeptide in cells, tissues, subjects, and samples (e.g., from subjects, in culture, etc.), include, but are not limited to: binding assays, including specific binding assays such as using antibodies or antigen-binding fragments thereof of the invention that bind specifically to acetylated cytochrome c polypeptide; gel electrophoresis; mass spectrometry; NMR; and the like. Immunoassays may be used according to the invention including, but not limited to, sandwich-type assays, competitive binding assays, one-step direct tests and two-step tests, etc. Assessment of binding of antibodies that specifically bind acetylated cytochrome c may also be done in vivo—in living subjects using art-known detectable labels and suitable in vivo methods.

Methods and assays of the invention (e.g., binding assays, gel electrophoresis; mass spectrometry; NMR; and the like) may be used to monitor changes in cytochrome c acetylation levels in a cell sample and or a subject over time, or changes in acetylation of specific residues of cytochrome c in a cell sample and or a subject over time. Methods for measuring acetylation of cytochrome c described herein can be applied to methods for screening for modulators of sirtuin activity, such as SIRT3 activity, as described above.

### Mass Spectrometry

Acetylation of cytochrome c may be measured by mass spectrometry. Mass spectrometry is an important tool in the identification of proteins and peptides, and in the identification of modified residues within proteins and peptides. In some embodiments, mass spectrometry is used to determine the level of acetylation of cytochrome c. In some embodiments mass spectrometry is used to determine whether cytochrome c is acetylated on specific residues.

Using mass spectrometry, such as ESI or MALDI-MS, peptides can be ionized intact into the gas phase and their masses accurately measured. Based on this information, proteins can readily be identified using protein mass mapping or peptide mass mapping, in which these measured masses are compared to predicted values derived from a protein database. Further sequence information can also be obtained by fragmenting individual peptides in tandem MS experiments.

Sequence specific proteases or certain chemical cleaving agents are used to obtain a set of peptides from the target protein that are then mass analyzed. The observed masses of the proteolytic fragments are compared with theoretical “in silico” digests of all proteins listed in sequence database. The matches or “hits” are then statistically evaluated and marked according to the highest probability.

Tandem mass spectrometry experiments allow peptide identification by yielding fragmentation patterns for individual peptide. Analogous to peptide mapping experiments, the experimentally obtained fragmentation patterns can be compared to theoretically generated MS/MS fragmentation patterns for the various proteolytic peptides arising from each protein contained in the searched database. Statistical evaluation of the results and scoring algorithms using search engines such a Sequest (ThermoFinnigan Corp) and MASCOT (Matrix Science, Limited) facilitate the identification of the best match. The partial sequence information contained in tandem MS experiments is more specific than simply using the mass of a peptide, since two peptides with identical amino acid contents but different sequences will exhibit different fragmentation patterns. Tandem mass spectrometry, the ability to induce fragmentation and perform successive mass spectrometry experiments on these ions, is generally used to obtain structural information through fragmentation.

One of the processes by which fragmentation is initiated is known as collision-induced dissociation (CID). CID is accomplished by selecting an ion of interest with the mass analyzer and then subjecting that ion to collisions with neutral atoms or molecules. The selected ion will collide with the collision gas such as argon, resulting in fragment ions which are then mass analyzed. CID can be accomplished with a variety of instruments, most commonly using triple quadrupoles, quadrupole ion traps, Fourier transform-ion cyclotron resonance (FT-ICR) mass spectrometry (FTMS), time-of-flight reflectron and quadrupole time-of-flight mass analyzers. The triple quadrupole and quadrupole ion trap combined with electrospray are common means of generating peptide structural data, as they are capable of high sensitivity, and produce a reasonable amount of fragmentation information. MALDI with time-of-flight reflectron and Fourier transform-ion cyclotron resonance are also common sources for structural information.

In order to obtain peptide sequence information by mass spectrometry, fragments of an ion must be produced that reflect structural features of the original compound. Most peptides are linear molecules, which allow for relatively straightforward interpretation of the fragmentation data. The process is initiated by converting some of the kinetic energy from the peptide ion into vibrational energy. This is achieved by introducing the selected ion, usually an (M+H)+ or (M+nH)ⁿ+ ion, into a collision cell where is collides with neutral Ar, Xe, or He atoms, resulting in fragmentation. The fragments are then monitored via mass analysis. Tandem mass spectrometry allows for a heterogeneous solution of peptides to be analyzed and then by filtering the ion of interest into the collision cell, structural information can be derived on each peptide from complex mixture.

Certain limitations for obtaining complete sequence information exist using tandem mass spectrometry. For example, in determining the amino acid sequence of a peptide, it is not possible for leucine and isoleucine to be distinguished because they have the same mass. The same difficulty will arise with lysine and glutamine since they have the same nominal mass, although high resolution tandem analyzers (quadrupole-TOF and FTMS) can distinguish between these amino acids.

In some preferred embodiments, samples of proteins (or peptides in a proteolytic digest) are separated by gel electrophoresis or liquid chromatography prior to mass analysis.

Gel electrophoresis is one of the most widely used techniques for separating intact proteins. In sodium dodecyl sulfate-polyacrylamide gel electrophoresis (SDS-PAGE), sometimes called one dimensional gel electrophoresis, the proteins are treated with the denaturing detergent SDS and loaded onto a gel. Upon application of an electric potential across the gel, the proteins migrate through the gel towards the anode at a rate inversely proportional to their size. Upon completion of the separation, the proteins may be visualized using any of a number of different staining agents (Coomassie, Sypro Ruby, or Silver), and the individual bands are physically excised from the gel. These excised spots are subjected to destaining, reductive alkylation, in-gel digestion, peptide extraction, and finally mass analysis for protein identification.

The combination of SDS-PAGE electrophoresis with an isoelectric focusing step also enables the separation of proteins of similar mass. In two-dimensional gel electrophoresis (2D-GE), proteins are first separated according to their isoelectric points (pI) by electrophoresis through a solution or gel containing an immobilized pH gradient, with each protein migrating to a position in the pH gradient corresponding to its isoelectric point. Once the isoelectric focusing step is complete, gel electrophoresis similar to SDS-PAGE is performed orthogonally to separate the proteins by size. Like 1D gels, 2D gel spots can be cut out, enzymatically digested, and mass analyzed for protein identification. Using this technique, thousands of proteins can simultaneously be separated and removed for identification.

Automated liquid handling robots have been developed that perform all the sample preparation steps for peptide mapping experiments, including gel destaining, alkylation/reduction, in gel digestion, peptide extraction, and MALDI target plating.

Mass spectral data acquisition systems have similarly been automated to acquire spectra, process the raw data, and perform database searches for numerous samples. Commercial MALDI-TOF systems are available that can perform over 1,000 mapping experiments in just twelve hours. These systems are able to perform automated calibrations, vary laser energies, and adjust laser firing location to maximize signal, with the entire data acquisition process requiring approximately 30 seconds or less. Similarly, automated data processing systems can recognize suitable signals, identify monoisotopic peaks, and submit summary peak lists directly to a search engine.

Such high throughput proteomics systems enable the investigation of multiple unknown samples at once such as those coming from gels. Additionally, the flexibility of automated acquisition and data analysis software allows to rapidly reacquire and/or reanalyze entire batches of samples with minimal user effort. Automated systems are, however, limited in that they are only as good as the data provided. For example, the detection and accurate mass assignment of species exhibiting low signal-to-noise ratios is often poor. Such issues have led to the development of post-acquisition data processing. Improvements in these processes have enabled high through-put automated systems to achieve identification “hit” rates equal to or above those obtained normally.

An alternative approach to gel electrophoresis techniques involves the use of analytical separation methods such as high performance liquid chromatography (HPLC). Whereas gel electrophoresis techniques separate intact proteins, liquid chromatography can be performed on proteolytic peptides. One of the means of performing peptide LC-MS/MS involves the direct coupling of the LC to an ion trap mass spectrometer through an electrospray ionization interface. Other mass analyzers suitable for these experiments include triple quadrupoles and quadrupole time-of-flights.

In some embodiments, mass spectrometry is used to determined whether cytochrome c is acetylated and on which specific residues cytochrome c is acetylated. The use of mass spectrometry to identify acetylation of lysine residues within proteins is discussed further in Zhang et al., (2002) Mol Cell Proteomics 1:500-508 and Dormeyer et al., (2005) Mol Cell Proteomics 4:1226-1239, incorporated herein by reference in their entirety.

### Diagnosis and Characterization of Risk of Neurodegenerative Disorders and Cancer

Methods and assays such as those discussed herein, for detecting acetylation of cytochrome c, allow monitoring of acetylated cytochrome c polypeptide levels in a subject who is believed to be at risk of a disorder associated with cytochrome c activity, and also enable monitoring in a subject who is known to have a disorder associated with cytochrome c activity.

Aspects of the invention relate to methods of diagnosing a neurodegenerative disorder characterized by acetylation of cytochrome c, or characterizing a subject's risk of a neurodegenerative disorder that is characterized by acetylation of cytochrome c. Further aspects of the invention relate to methods of diagnosing a cancer characterized by deacetylation of cytochrome c, or characterizing a subject's risk of a cancer that is characterized by deacetylation of cytochrome c.

Methods involve detecting the level of acetylation of cytochrome c polypeptide in a sample from a subject and comparing the level of acetylation of cytochrome c to a control sample or a predetermined value. The acetylation state of the protein may be determined by any of the methods described herein. Assays based upon detecting levels of acetylated cytochrome c in cells and/or subjects include determining onset, progression, and/or regression of a neurodegenerative disorder or a cancer in a subject; selecting a treatment for a neurodegenerative disorder or a cancer in a subject; and evaluating a treatment for cytochrome c polypeptide acetylation status in a subject. Thus, subjects can be characterized, treatment regimens can be monitored, treatments can be selected and diseases status can be better understood using the assays of the present invention. The level of acetylated cytochrome c polypeptide may correlate with the status of a neurodegenerative disorder or a cancer in a subject.

One aspect of the present invention relates to detecting acetylated cytochrome c polypeptides or fragments thereof in an in vitro or in vivo sample (e.g., histological or cytological specimens, real-time in vivo assays, biopsies and the like), and, in particular, to distinguish the level of acetylated cytochrome c from the level of non-acetylated cytochrome c in a sample or a subject. In some embodiment, this method involves providing an antibody or an antigen-binding binding fragment thereof, which specifically binds to acetylated cytochrome c polypeptide. The anti-acetylated cytochrome c antibody may be bound to a label that permits the detection of the acetylated cytochrome c polypeptide. In some embodiments, a sample may be contacted with a labeled anti-acetylated cytochrome c antibody under conditions effective to permit binding of the anti-acetylated cytochrome c antibody to acetylated cytochrome c polypeptide in the sample. The presence of acetylated cytochrome c in a sample may be detected by detection of the label. In some embodiments, the contact between the anti-acetylated cytochrome c antibody and a sample is carried out in samples from a subject. In certain embodiments, the contact between an anti-acetylated cytochrome c antibody and a sample may be carried out in a subject. Samples to which the methods of the invention can be applied include tissue samples, cell samples, including cell culture samples, subject samples, in vivo samples, etc. In some embodiments mass spectrometry is used to identify acetylation of cytochrome c.

Assays to detect acetylation of cytochrome c may be carried out in cells from culture, cells in solution, in samples obtained from subjects, and/or samples in a subject (in vivo sample). As used herein, a subject is a human, non-human primate, cow, horse, pig, sheep, goat, dog, cat, or rodent. In some embodiments, human subjects are preferred. The samples used herein include any cell or tissue sample, and may include neuronal cell and/or tissue samples.

Particularly important subjects to which the present invention can be applied are subjects with a neurodegenerative disorder. The term “subject with a neurodegenerative disorder” as used herein, means an individual who, at the time the sample is taken, has been diagnosed as having a neurodegenerative disorder. Methods of the invention may also be used to detect abnormal levels of cytochrome c polypeptide acetylation in subjects that are not yet diagnosed with a neurodegenerative disorder. The onset, progression, and/or regression of a neurodegenerative disorder may also be monitored using methods and antibodies of the invention.

Particularly important subjects to which the present invention can be applied are subjects with a cancer. The term “subject with a cancer” as used herein, means an individual who, at the time the sample is taken, has been diagnosed as having a cancer. Methods of the invention may also be used to detect abnormal levels of cytochrome c polypeptide acetylation in subjects that are not yet diagnosed with a cancer. The onset, progression, and/or regression of a cancer may also be monitored using methods and antibodies of the invention.

In some embodiments, aspects of the invention relate to screening subjects for diseases associated with the presence of elevated levels of acetylated cytochrome c polypeptide. As used herein, the term “elevated” means higher, for example elevated versus a control level. In some embodiments, the status and/or stage of a neurodegenerative disorder is determined by assessing the level of acetylated cytochrome c in a sample from a subject or culture that has a neurodegenerative disorder. Antibodies of the invention are useful in assays to differentiate whether or not a subject has a neurodegenerative disorder, because anti-acetylated cytochrome c antibodies of the invention can be used to quantitate the amount of acetylated cytochrome c polypeptide in cells and tissues of subjects who have neurodegenerative disorders, or who are at risk of having neurodegenerative disorders. As discussed above, mass spectrometry approaches can also be used to identify specific residues of cytochrome c that are acetylated in a sample from a subject. The presence of acetylated cytochrome c polypeptide in a sample, and/or the detection of acetylation of specific residues of cytochrome c in a sample, can be used to determine the presence and/or status of a neurodegenerative disorder in a cell, cell culture or subject. Methods of the invention can be used to obtain useful prognostic information by providing an early indicator of disease onset and/or progression. In some embodiments, the disorder is a cancer and the subject exhibits decreased levels of acetylated cytochrome c or increased levels of deacetylated cytochrome c.

Levels of acetylated cytochrome c polypeptide (e.g., K40- or K74-acetylated cytochrome c polypeptide) can be determined in a number of ways when carrying out the various methods of the invention. In one measurement, a level of acetylated cytochrome c polypeptide is measured in relation to non-acetylated (or deacetylated) cytochrome c polypeptide. Thus, the measurement may be a relative measure, which can be expressed, for example, as a percentage of total cytochrome c polypeptide. Those of ordinary skill in the art will appreciate that relative amounts of acetylated and non-acetylated cytochrome c polypeptides may be determined by measuring either the relative amount of acetylated cytochrome c polypeptide or the relative amount of non-acetylated cytochrome c polypeptide. In other words, if 90% of an individual's cytochrome c polypeptide is non-acetylated cytochrome c polypeptide (or reduced acetylated cytochrome c polypeptide), then 10% of the individual's cytochrome c polypeptide will be acetylated cytochrome c polypeptide.

Another measurement of the level of acetylated cytochrome c is a measurement of absolute levels of cytochrome c polypeptide acetylation. This could be expressed, for example, in acetylated cytochrome c polypeptide per unit of cells or tissue. Another measurement of the level of acetylated cytochrome c polypeptide is a measurement of the change in the level of acetylated cytochrome c polypeptide over time. This may be expressed in an absolute amount or may be expressed in terms of a percentage increase or decrease over time.

Aspects of the invention relate to characterizing cytochrome c polypeptide acetylation levels by monitoring changes in the absolute or relative amounts of acetylated cytochrome c polypeptide in a subject or sample (e.g., a cell culture) over time. In some embodiments, changes in relative or absolute acetylated cytochrome c polypeptide of greater than 0.1% may indicate an abnormality. Preferably, the change in acetylated cytochrome c polypeptide levels that indicates an abnormality, is greater than 0.2%, greater than 0.5%, greater than 1.0%, 2.0%, 3.0%, 4.0%, 5.0%, 7.0%, 10%, 15%, 20%, 25%, 30%, 40%, 50%, or more.

Levels of acetylated cytochrome c polypeptide can be determined and are compared to controls according to the invention. The control may be a predetermined value, which can take a variety of forms. It can be a single cut-off value, such as a median or mean. It can be established based upon comparative groups, such as in groups having normal amounts of cytochrome c acetylation and groups having abnormal amounts of cytochrome c acetylation. Another example of comparative groups may be groups having symptoms of a neurodegenerative disorder and groups without symptoms of a neurodegenerative disorder. Another comparative group may be a group with a family history of a neurodegenerative disorder and a group without such a family history. In some embodiments, the risk in one defined group is double the risk in another defined group. A predetermined value can be arranged, for example, where a tested population is divided equally (or unequally) into groups, such as a low-risk group, a medium-risk group and a high-risk group or into quadrants or quintiles, the lowest quadrant or quintile being individuals with the lowest risk and lowest amounts of acetylated cytochrome c polypeptide and the highest quadrant or quintile being individuals with the highest risk and highest amounts of acetylated cytochrome c polypeptide.

The predetermined value, of course, will depend upon the particular population selected. For example, an apparently healthy population will have a different ‘normal’ range than will a population that is known to have a condition related to abnormal cytochrome c polypeptide acetylation. Accordingly, the predetermined value selected may take into account the category in which an individual or cell falls. Appropriate ranges and categories can be selected with no more than routine experimentation by those of ordinary skill in the art. As used herein, “abnormal” means not normal as compared to a control. By abnormally high it is meant high relative to a selected control. Typically the control will be based on apparently healthy normal individuals in an appropriate age bracket or apparently healthy cells.

It will also be understood that controls according to the invention may be, in addition to predetermined values, samples of materials tested in parallel with the experimental materials. Examples include samples from control populations or control samples generated through manufacture to be tested in parallel with the experimental samples.

### Evaluating Efficacy of Therapy

Methods of the invention may also be used to assess the efficacy of a therapeutic treatment of a neurodegenerative disorder or a cancer and for the assessment of the level of acetylated cytochrome c polypeptide a subject at various time points. For example, a level of a subject's acetylated cytochrome c polypeptide can be obtained prior to the start of a therapeutic regimen (either prophylactic or as a treatment of a neurodegenerative disorder or a cancer), during the treatment regimen and/or after a treatment regimen, thus providing information on the effectiveness of the regimen in the patient. Assessment of efficacy of candidate therapeutic agents may also be done using assays of the invention in cells from culture—e.g., as screening assays to assess candidate therapeutic agents.

It will be understood that a therapeutic regimen may be either prophylactic or a treatment of a neurodegenerative disorder or a cancer in a subject. Thus, methods of the invention may be used to monitor a subject's response to prophylactic therapy and/or treatment for a neurodegenerative disorder or a cancer provided to a subject. Methods of the invention (e.g., binding assays, gel electrophoresis; mass spectrometry; NMR; and the like) may also be useful to monitor the onset, progression, or regression of a neurodegenerative disorder or a cancer in a subject. The level of acetylated cytochrome c polypeptide may be determined in two, three, four, or more samples obtained from a subject at separate times. The level of acetylated cytochrome c polypeptide in the samples may be compared and changes in the levels over time may be used to assess the status and stage of a neurodegenerative disorder or a cancer in a subject and/or the effect of a treatment strategy on the neurodegenerative disorder or a cancer in a subject.

Aspects of the invention relate to monitoring therapy or evaluating efficacy of therapy in a subject. The method involves obtaining a level of acetylated cytochrome c in a subject undergoing therapy. The level of acetylated cytochrome c is compared to a predetermined value corresponding to a control level of acetylated cytochrome c (e.g., in an apparently healthy population). A determination of whether the level of acetylated cytochrome c is at, below or above a predetermined level will contribute to an indication of whether the subject would benefit from continued therapy with the same therapy or would benefit from a change in therapy. Health care practitioners select therapeutic regimens for treatment based upon the expected net benefit to the subject. The net benefit is derived from the risk to benefit ratio. The present invention permits the determination of whether a subject will benefit from continued therapy or would benefit from a change in therapy, thereby aiding the physician in selecting a therapy. The benefit is typically a reduction in the signs and symptoms or complications of a neurodegenerative disorder or a cancer. Signs, symptoms, manifestations and complications of neurodegenerative disorders and cancers are known to those of ordinary skill in the art. In some embodiments, a determination that the level of acetylated cytochrome c is at or below a predetermined level will indicate that the subject would benefit from continued therapy with the same therapy. In some embodiments, a determination that the level of acetylated cytochrome c is at or above a predetermined level indicates that the subject would benefit from change in therapy. In some embodiments, obtaining a level of acetylated cytochrome c is repeated so as to monitor the subject's levels of acetylated cytochrome c over time.

In some embodiments, the subject may have been undergoing the therapy for at least 1, 2, 3, 4, 5, 6, 7 days or more. In some embodiments, the subject may have been undergoing the therapy for at least 1, 2, 3, 4, 5, 6, 7, 8, 9, 10, 11, 12 weeks or more. In some embodiments, the subject may have been undergoing the therapy for at least 3, 4, 5, 6, 7, 8, 9, 10, 11, 12 months or more.

In some embodiments, a subject who would benefit from continued therapy is a subject whose on-therapy level of acetylated cytochrome c reaches a certain predetermined value or whose level of acetylated cytochrome c is decreasing. In some embodiments, a subject who would benefit from a change in therapy is a subject whose on-therapy level of acetylated cytochrome c did not reach a certain predetermined value or whose on-therapy level of acetylated cytochrome c is not decreasing.

In some embodiments, a subject who would benefit from continued therapy is a subject whose on-therapy level of acetylated cytochrome c reaches a certain predetermined value or whose level of acetylated cytochrome c is increasing. In some embodiments, a subject who would benefit from a change in therapy is a subject whose on-therapy level of acetylated cytochrome c did not reach a certain predetermined value or whose on-therapy level of acetylated cytochrome c is not increasing.

As used herein, a “change in therapy” refers to an increase or decrease in the dose of the existing therapy, a switch from one therapy to another therapy, an addition of another therapy to the existing therapy, or a combination thereof. A switch from one therapy to another may involve a switch to a therapy with a high risk profile but where the likelihood of expected benefit is increased. In some embodiments, preferred therapies are therapies that decrease the level(s) of acetylated cytochrome c. In some embodiments, preferred therapies are therapies that increase the level(s) of acetylated cytochrome c. A subject who would benefit from a change in therapy by increasing the dose of the existing therapy is a subject who, for example, was on the therapy but was not receiving the maximum tolerated dose or the maximum allowed dose of the therapy and whose level of acetylated cytochrome c did not reach a certain predetermined value. In such instances the dose of the existing therapy is increased until the level of acetylated cytochrome c reaches a certain predetermined value. In some instances, the dose of the existing therapy is increased from the existing dose to a higher dose that is not the maximum tolerated dose nor the maximum allowed dose of the therapy. In other instances, the dose is increased to the maximum tolerated or to the maximum allowed dose of the therapy. A subject who would benefit from a change in therapy by decreasing the dose of the existing therapy is, for example, a subject whose on-therapy level of acetylated cytochrome c reaches or can reach a certain predetermined value with a lower dose of the therapy.

A subject who would benefit from a switch from one therapy to another therapy is, for example, a subject who was on the maximum tolerated dose or the maximum allowed dose of the therapy and whose level of deacetylated cytochrome c did not reach a certain predetermined value. Another example is a subject was not on the maximum tolerated or the maximum allowed dose of the therapy but was determined by a health care practitioner to more likely benefit from another therapy. Such determinations are based, for example, on the development in the subject of unwanted side effects on the initial therapy or a lack of response to the initial therapy.

A subject who would benefit from a change in therapy by the addition of another therapy to the existing therapy is, for example, a subject who was on a therapy but whose level of acetylated cytochrome c did not reach a certain predetermined value. In such instances, another therapy is added to the existing therapy. The therapy that is added to the existing therapy can have a different mechanism of action in decreasing the level of acetylated cytochrome c than the existing therapy. In some instances, a combination of the aforementioned changes in therapy may be used.

Those of ordinary skill in the art will recognize that similar assessments of candidate therapeutics can be tested in vitro by assessing any change in cytochrome c acetylation that occurs in response to contact of the cell with a candidate agent for treatment of a neurodegenerative disorder.

Aspects of the invention relate to the measurement of acetylated cytochrome c levels to guide treatments in order to improve outcome in subjects. Levels of acetylated cytochrome c have predictive value for response to treatments to reduce the risk of mortality in a subject with a neurodegenerative disorder or a cancer. Subjects who would benefit from this aspect of this invention are subjects who are undergoing therapy to reduce the risk of mortality (e.g., from a neurodegenerative disorder or a cancer). A subject on-therapy is a subject who already has been diagnosed with a neurodegenerative disorder or a cancer and is in the course of treatment with a therapy. The therapy can be any of the therapeutic agents used in the treatment of neurodegenerative disorders or cancers. Therapeutic agents used in the treatment of neurodegenerative disorders or cancers are known to those of ordinary skill in the art. The therapy also can be non-drug treatments. In some embodiments, the therapy is one which decreases levels of acetylated cytochrome c or increases levels of deacetylated cytochrome c. In some embodiments, the therapy is one which increases levels of acetylated cytochrome c or decreases levels of deacetylated cytochrome c. Methods associated with the invention for identifying acetylation status of cytochrome c can be used to obtain measurements that represent the diagnosis of a neurodegenerative disorder or a cancer in a subject. In some instances, a subject may be already be undergoing drug therapy for a neurodegenerative disorder or a cancer, while in other instances a subject may be without present therapy for the neurodegenerative disorder or cancer.

The amount of a treatment may be varied for example by increasing or decreasing the amount of a pharmacological agent or a therapeutic composition, by changing the therapeutic composition administered, by changing the route of administration, by changing the dosage timing and so on.

### Selecting a Subject for Treatment

As used herein, the term treat, treated, or treating when used with respect to a disorder refers to a prophylactic treatment that increases the resistance of a subject to development of the disease or, in other words, decreases the likelihood that the subject will develop the disease as well as a treatment after the subject has developed the disease in order to fight the disease or prevent the disease from becoming worse. The term “treatment” embraces the prevention of a disorder or condition, and the inhibition and/or amelioration of pre-existing disorders and conditions. A subject may receive treatment because the subject has been determined to be at risk of developing a disorder or condition, or alternatively, the subject may have such a disorder or condition. Thus, a treatment may prevent, reduce or eliminate a disorder or condition altogether or prevent it from becoming worse.

As used herein, the term “subject” refers to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments of the invention, a subject is a patient. As used herein, a “patient” refers to a subject who is under the care of a physician or other health care worker, including someone who has consulted with, received advice from or received a prescription or other recommendation from a physician or other health care worker.

Aspects of the invention relate to selecting subjects for treatment who have abnormal levels of acetylated cytochrome c polypeptide. Treatment may include administration of an agent that will mediate acetylation or deacetylation of cytochrome c. Such subjects may already be receiving a drug for treating a neurodegenerative disorder or a cancer. In some embodiments, a subject may be free of any present treatment for a neurodegenerative disorder but monitoring of cytochrome c polypeptide acetylation levels using methods and/or antibodies of the invention, may identify the subject as a candidate for a treatment to increase deacetylation of cytochrome c and/or treatment to decrease acetylation of cytochrome c polypeptide. In some embodiments, a subject may be free of any present treatment for a cancer but monitoring of cytochrome c polypeptide acetylation levels using methods and/or antibodies of the invention, may identify the subject as a candidate for a treatment to increase acetylation of cytochrome c and/or treatment to decrease deacetylation of cytochrome c polypeptide. Thus, subjects may be selected and treated with elevated levels of the same drugs or with different therapies as a result of assays that determine the acetylation status of cytochrome c.

According to the present invention, some subjects may be free of symptoms otherwise calling for treatment with a particular therapy, and testing with methods of the invention such as an anti-cytochrome c polypeptide-acetylation antibody may identify the subject as needing treatment. This means that absent the use of the antibodies or antigen-binding fragments thereof of the invention to assess levels of acetylated cytochrome c polypeptide, the subject would not according to convention as of the date of the filing of the present application have symptoms calling for treatment with a particular therapy. As a result of measuring the level of acetylated cytochrome c polypeptide that the subject that a subject has, the subject become a candidate for treatment with the therapy.

### Treatment

According to still another aspect of the invention, compounds that decrease the level of acetylation of cytochrome c may be administered to inhibit apoptosis in a cell, and to prevent and/or treat a neurodegenerative disorder. In some embodiments, peptides or polypeptides of cytochrome c containing deacetylated K40 and K74 residues can be administered.

Compounds useful to decrease levels of acetylation of cytochrome c and which may be administered as a treatment for neurodegenerative disorders include, but are not limited to deacetylase proteins. In some embodiments the deacetylase protein is a sirtuin. In some embodiments the sirtuin is SIRT3.

In a subject determined to have an abnormally high level of acetylation of cytochrome c polypeptide, a treatment (e.g., a compound that decreases the level of acetylation of cytochrome c polypeptide) is that amount effective to decrease the level of acetylation of cytochrome c in the subject or increase the amount of deacetylation in the subject—each of which will decrease the level of acetylated cytochrome c polypeptide relative to the level that was present prior to treatment. Thus, compounds that increase deacetylation levels of cytochrome c polypeptides (e.g., SIRT3) may be administered in effective amounts to inhibit apoptosis and to prevent and/or treat a neurodegenerative disorder. Typically an effective amount of a compound that decreases a level of acetylated cytochrome c (e.g., SIRT3 or a compound that increases the expression or activity of SIRT3) will be determined in clinical trials, establishing an effective dose for a test population versus a control population in a blind study. In some embodiments, an effective amount will be an amount that results in a desired response, e.g., an amount that diminishes or eliminates symptoms of a neurodegenerative disorder. In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

Effective amounts of therapeutic compounds or compositions (each of which may be referred to herein as pharmaceutical or therapeutic compounds or compositions) may also be determined by assessing physiological effects of administration on a cell or subject, such as a decrease of disease symptoms following administration. Other assays will be known to one of ordinary skill in the art and can be employed for measuring the level of the response to a treatment. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount may depend upon the degree to which an individual has abnormally elevated levels of acetylation of cytochrome c polypeptide.

A pharmaceutical compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

The absolute amount will depend upon a variety of factors, including the material selected for administration, whether the administration is in single or multiple doses, and individual subject parameters including age, physical condition, size, weight, and the stage of the disease or condition. These factors are well known to those of ordinary skill in the art and can be addressed with no more than routine experimentation.

Further aspects of the invention relate to inducing apoptosis in a cell that exhibits deacetylated cytochrome c. As discussed above, acetylated cytochrome c is associated with apoptosis. Thus contacting a cell with an agent that induces acetylation or reduces deacetylation of cytochrome c represents a method for inducing apoptosis in a cell. In some embodiments the disease or condition associated with deacetylated cytochrome c is cancer. In some embodiments a cancer patient is selected for treatment and treated with an agent or composition that acetylates cytochrome c or prevents deacetylation of cytochrome c, if the cancer patient has a cancer that exhibits deacetylation of lysine residues corresponding to residues K40 and/or K74 in a full-length, wild-type polypeptide. In some embodiments, peptides or polypeptides of cytochrome c containing acetylated K40 and K74 residues can be administered.

### Antibodies

The invention includes in one aspect, antibodies that specifically bind synthetic and natural acetylated cytochrome c, and methods for their preparation and use. The invention includes, in part, methods for preparing acetylated cytochrome c polypeptides, including, but not limited to K40- and K-74-acetylated cytochrome c polypeptides. Acetylated cytochrome c polypeptides may be used as antigens to make antibodies that specifically bind acetylated cytochrome c polypeptide. Compositions useful for making an antibody of the invention may include an acetylated cytochrome c polypeptide molecule. In some embodiments, an acetylated cytochrome c polypeptide or fragment thereof may be an acetylated full-length, wild-type or mutant cytochrome c polypeptide, or a fragment of a wild-type or mutant full-length cytochrome c that is an acetylated fragment.

Methods of the invention may also include the use of fragments of cytochrome c polypeptides for the production of antibodies that specifically bind acetylated cytochrome c polypeptides. In some embodiments, an acetylated lysine residue of a cytochrome c polypeptide that is part of the epitope specifically recognized by the antibody is a lysine residue that corresponds to an acetylated residue of wild-type, full-length cytochrome c polypeptide. In some embodiments, an acetylated residue corresponds to residue K40 or K74 of wild-type, full-length human cytochrome c polypeptide. In some embodiments, an antigenic polypeptide can be as small as 5 amino acids in length. In some embodiments, when the size of the polypeptide antigen is less than about 8 amino acids in length, a second carrier molecule, e.g., bovine serum albumin (BSA), may be attached to the polypeptide to increase antigenicity of the polypeptide. Thus, small fragments of cytochrome c that include the desired epitope for antibody production can be used in the production of an antibody that specifically binds to the epitope, which includes an acetylated lysine residue (e.g., a K40- or K74-acetylated residue).

Any cytochrome c polypeptide fragment that includes an acetylated lysine residue may be used in conjunction with a second molecule, e.g., keyhole limpet hemocyanin (KLH) or bovine serum albumin (BSA) as described above, as an antigenic polypeptide with which to prepare antibodies that specifically bind to a cytochrome c acetylated polypeptide. In some embodiments, an antigenic polypeptide may be a cytochrome c polypeptide fragment that includes acetylated K40 and/or K74, and an antibody generated from such an antigen will specifically bind to a K40- or K74-acetylated epitope of cytochrome c polypeptide. Anti-cytochrome c polypeptide antibodies or antigen-binding fragments thereof may be purified using art-known affinity purification and/or affinity selection methods. Affinity selection is selection of antibodies or antigen-binding fragments thereof for binding to the target material (e.g., an acetylated cytochrome c polypeptide).

It will be understood by those of ordinary skill in the art that it is preferable that a fragment of cytochrome c polypeptide for use as an immunogenic fragment in the methods of the invention be at least 5, 6, 7, 8, 9, 10, 11, 12, 13, 14, 15, 16, 17, 18, 19, 20 or more amino acids in length. If a fragment of cytochrome c polypeptide includes more than one lysine residue, it is desirable that in some embodiments, only one of the lysine residues is an acetylated lysine residue. One of ordinary skill in the art will be able to use the guidance provided herein to make fragments of cytochrome c polypeptide that can be used in methods of the invention. In some embodiments the fragment of cytochrome c used to generate an antibody contains an acetylated lysine residue corresponding to residue K40 in a wild-type, full length human cytochrome c polypeptide. In some embodiments the fragment of cytochrome c used to generate an antibody contains an acetylated lysine residue corresponding to residue K74 in a wild-type, full length human cytochrome c polypeptide. In some embodiments the fragment of cytochrome c used to generate an antibody contains more than one acetylated lysine residues.

As used herein, the term “antibody” refers to a protein that may include at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen (e.g., acetylated cytochrome c polypeptide and in some embodiments, the acetylated cytochrome c polypeptide is K40- or K74-acetylated cytochrome c polypeptide or corresponding residue in a cytochrome c polypeptide fragment). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain or the variable domain of a heavy-chain antibody, such as a camelid heavy-chain antibody (e.g. V_{HH}); (vi) an isolated complementarity determining region (CDR); and (vii) polypeptide constructs comprising the antigen-binding fragments of (i)-(vi). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art, such as expression of recombinant nucleic acids. The fragments are screened for utility in the same manner as are intact antibodies.

Isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g., IgM or IgG1) that is encoded by heavy chain constant region genes. Antibodies of the invention can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

Antibodies of the present invention can be polyclonal, monoclonal, or a mixture of polyclonal and monoclonal antibodies. Antibodies of the invention can be produced by methods disclosed herein or by a variety of techniques known in the art. In some embodiments, the epitope recognized by an antibody of the invention includes acetylated lysine that corresponds to the K40 and/or K74 in full-length, wild-type cytochrome c polypeptide. In some embodiments, the epitope recognized by an antibody of the invention comprises an acetylated residue that corresponds to K40 and/or K74 of wild-type, full-length cytochrome c polypeptide.

Polyclonal and monoclonal antibodies may be prepared using techniques that are known in the art. The term “monoclonal antibody,” as used herein, refers to a preparation of antibody molecules of single molecular composition. A monoclonal antibody displays a single binding specificity and affinity for a particular epitope. A monoclonal antibody displays a single binding specificity and affinity for a particular epitope. The term “polyclonal antibody” refers to a preparation of antibody molecules that comprises a mixture of antibodies active that specifically bind a specific antigen.

A process of monoclonal antibody production may include obtaining immune somatic cells with the potential for producing antibody, in particular B lymphocytes, which have been previously immunized with the antigen of interest either in vivo or in vitro and that are suitable for fusion with a B-cell myeloma line. Mammalian lymphocytes typically are immunized by in vivo immunization of the animal (e.g., a mouse) with the desired protein or polypeptide, e.g., with acetylated cytochrome c polypeptide or a fragment thereof, or K40- or K74-acetylated cytochrome c or a fragment thereof in the present invention. In some embodiments, the polypeptide is a modified polypeptide as described herein. Such immunizations are repeated as necessary at intervals of up to several weeks to obtain a sufficient titer of antibodies. Once immunized, animals can be used as a source of antibody-producing lymphocytes which can be cloned and recombinantly expressed, as discussed further below. Following the last antigen boost, the animals are sacrificed and spleen cells removed. Mouse lymphocytes give a higher percentage of stable fusions with the mouse myeloma lines described herein. Of these, the BALB/c mouse is preferred. However, other mouse strains, rat, rabbit, hamster, sheep, goats, camels, llamas, frogs, etc. may also be used as hosts for preparing antibody-producing cells. See; Goding (in Monoclonal Antibodies: Principles and Practice, 2d ed., pp. 60-61, Orlando, Fla., Academic Press, 1986). Mouse strains that have human immunoglobulin genes inserted in the genome (and which cannot produce mouse immunoglobulins) can also be used. Examples include the HuMAb mouse strains produced by Medarex/GenPharm International, and the XenoMouse strains produced by Abgenix. Such mice produce fully human immunoglobulin molecules in response to immunization.

Those antibody-producing cells that are in the dividing plasmablast stage fuse preferentially. Somatic cells may be obtained from the lymph nodes, spleens and peripheral blood of antigen-primed animals, and the lymphatic cells of choice depend to a large extent on their empirical usefulness in the particular fusion system. The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include, but are not limited to Ag8, P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elsevier, 1984). Those of ordinary skill in the art will be aware of numerous routine methods to produce monoclonal antibodies.

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference). Methods of raising polyclonal antibodies are well known to those of ordinary skill in the art. As a non-limiting example, anti-acetylated cytochrome c polyclonal antibodies may be raised by administering an acetylated cytochrome polypeptide subcutaneously to New Zealand white rabbits which have first been bled to obtain pre-immune serum. The acetylated cytochrome can be inoculated with (e.g., injected at) a total volume of 100 μl per site at six different sites, typically with one or more adjuvants. The rabbits are then bled two weeks after the first injection and periodically boosted with the same antigen three times every six weeks. A sample of serum is collected 10 days after each boost. Polyclonal antibodies are recovered from the serum, preferably by affinity chromatography using acetylated cytochrome to capture the antibody. This and other procedures for raising polyclonal antibodies are disclosed in E. Harlow, et al., editors, Antibodies: A Laboratory Manual (1988), which is hereby incorporated by reference. Those of ordinary skill in the art will be aware of numerous routine methods to produce polyclonal antibodies. In some embodiments, the epitope recognized by the polyclonal antibody of the invention comprises an acetylated residue that corresponds to K40 or K74 of wild-type, full-length cytochrome c polypeptide.

In other embodiments, antibodies may be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, genetically engineered antibodies, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

The present invention further provides nucleic acid molecules encoding anti-acetylated cytochrome c antibodies (e.g., anti-K40- or K74-acetylated cytochrome c antibodies) and vectors comprising the nucleic acid molecules as described herein. The vectors provided can be used to transform or transfect host cells for producing anti-acetylated cytochrome c antibodies with the specificity of antibodies described herein. In some embodiments, the vectors can include an isolated nucleic acid molecule encoding a heavy chain and/or a light chain of an antibody of the invention encoded by a nucleic acid molecule. In a further embodiment, plasmids are given which produce the antibodies or antigen-binding fragments described herein.

Antibodies or antigen-binding fragments of the invention are, preferably, isolated. “Isolated”, as used herein with respect to antibodies and antigen-binding fragments thereof, is intended to refer to an antibody (or antigen-binding fragment thereof) that is substantially free of other antibodies (or antigen-binding fragments) having different antigenic specificities (e.g., an isolated antibody that specifically binds to acetylated cytochrome c polypeptide is substantially free of antibodies that specifically bind antigens other than acetylated cytochrome c polypeptide). An isolated antibody that specifically binds to an epitope, isoform or variant of a acetylated polypeptide (e.g., acetylated cytochrome c polypeptide) may, however, have cross-reactivity to other related antigens, e.g., a mutant form of cytochrome c, or a polypeptide from other species (e.g., cytochrome c species homologs). Moreover, an isolated antibody (or antigen-binding fragment thereof) may be substantially free of other cellular material and/or chemicals.

Antibodies of the invention include, but are not limited to antibodies that specifically bind to an acetylated cytochrome c polypeptide. In certain embodiments, an antibody of the invention specifically binds cytochrome c that is acetylated at residues that correspond to the K40 and/or K74 residue of full-length, wild-type cytochrome c polypeptide. As used herein, “specific binding” refers to antibody binding to a predetermined antigen with a preference that enables the antibody to be used to distinguish the antigen from others to an extent that permits the diagnostic and other assays described herein. Specific binding to K40- or K74-acetylated cytochrome c polypeptide means that the antibody not only preferentially binds cytochrome c polypeptide versus other polypeptides, but also that it preferentially binds an acetylated cytochrome c polypeptide versus a cytochrome c polypeptide that is not acetylated. Typically, the antibody binds with an affinity that is at least two-fold greater than its affinity for binding to antigens other than the predetermined antigen. In some embodiments, an antibody or antigen-binding fragment thereof of the invention specifically binds to K40- or K74-acetylated cytochrome c polypeptide. It will be understood that the cytochrome c polypeptide or fragment thereof that includes an acetylated residue that corresponds to acetylated K40 or K74 of full-length, wild-type cytochrome c polypeptide, may be a wild-type or a mutant form of cytochrome c polypeptide—as long as the epitope recognized by an antibody that specifically binds an acetylated cytochrome c polypeptide residue that includes a residue corresponding to acetylated K40 or K74 residue of full-length, wild-type cytochrome c polypeptide is present.

Anti-K40- or K74-acetylated cytochrome c antibodies or antigen-binding fragments thereof, of the invention, can specifically bind K40- or K74-acetylated cytochrome c polypeptide with sub-nanomolar affinity. The binding affinities can be about 1×10⁻⁶, 1×10⁻⁷, 1×10⁻⁸, 1×10⁻⁹M or less, preferably about 1×10⁻¹⁰ M or less, more preferably 1×10⁻¹¹M or less. In a particular embodiment the binding affinity is less than about 5×10⁻¹⁰M.

In some aspects of the invention, an antibody or antigen-binding fragment thereof binds to a conformational epitope within the acetylated cytochrome c polypeptide. To determine if the selected anti-acetylated cytochrome c antibodies bind to conformational epitopes, each antibody can be tested in assays using native protein (e.g., non-denaturing immunoprecipitation, flow cytometric analysis of cell surface binding) and denatured protein (e.g., Western blot, immunoprecipitation of denatured proteins). A comparison of the results will indicate whether the antibodies bind conformational epitopes. Antibodies that bind to native protein but not denatured protein are those antibodies that bind conformational epitopes, and are preferred antibodies.

In some embodiments of the invention, antibodies competitively inhibit the specific binding of a second antibody to its target acetylated epitope on acetylated cytochrome c polypeptide. In some embodiments, the target epitope comprises an acetylated residue that corresponds to K40 or K74 of wild-type, full-length cytochrome c polypeptide. To determine competitive inhibition, a variety of assays known to one of ordinary skill in the art can be employed. For example, competition assays can be used to determine if an antibody competitively inhibits binding to acetylated cytochrome c (or K40- or K74-acetylated cytochrome c) by another antibody. These methods may include cell-based methods employing flow cytometry or solid phase binding analysis. Other assays that evaluate the ability of antibodies to cross-compete for acetylated cytochrome c polypeptide (or K40- or K74-acetylated cytochrome c polypeptide) molecules in solid phase or in solution phase, also can be used.

Certain antibodies competitively inhibit the specific binding of a second antibody to its target epitope on acetylated cytochrome c polypeptide (or K40- or K74-acetylated cytochrome c polypeptide) by at least about 10%, 20%, 30%, 40%, 50%, 60%, 70%, 80%, 90%, 95%, or 99%. Inhibition can be assessed at various molar ratios or mass ratios; for example competitive binding experiments can be conducted with a 2-fold, 3-fold, 4-fold, 5-fold, 7-fold, 10-fold or more molar excess of the first antibody over the second antibody.

Other antibodies of the invention may include antibodies that specifically bind to an epitope on acetylated cytochrome c polypeptide defined by a second antibody. To determine the epitope, one can use standard epitope mapping methods known in the art. For example, fragments (polypeptides) of K40- or K74-acetylated cytochrome c polypeptide antigen that bind the second antibody can be used to determine whether a candidate antibody binds the same epitope. In some embodiments, an epitope comprises an acetylated residue that corresponds to K40 or K74 of wild-type, full-length cytochrome c polypeptide. For linear epitopes, overlapping polypeptides of a defined length (e.g., 5, 6, 7, 8 or more amino acids) may be synthesized. The polypeptides preferably are offset by 1 amino acid, such that a series of polypeptides covering every 4, 5, 6, 7, or 8 amino acid fragment (respectively) of the acetylated cytochrome c polypeptide sequence are prepared. Fewer polypeptides can be prepared by using larger offsets, e.g., 2 or 3 amino acids. In addition, longer polypeptides (e.g., 9-, 10- or 11-mers) can be synthesized. Binding of polypeptides to antibodies can be determined using standard methodologies including surface plasmon resonance (BIACORE) and ELISA assays. For examination of conformational epitopes, larger acetylated cytochrome c polypeptide fragments, including in some embodiments K40- or K74-acetylated cytochrome c polypeptide, can be used. Other methods that use mass spectrometry to define conformational epitopes have been described and can be used (see, e.g., Baerga-Ortiz et al., Protein Science 11:1300-1308, 2002 and references cited therein). Still other methods for epitope determination are provided in standard laboratory reference works, such as Unit 6.8 (“Phage Display Selection and Analysis of B-cell Epitopes”) and Unit 9.8 (“Identification of Antigenic Determinants Using Synthetic Polypeptide Combinatorial Libraries”) of Current Protocols in Immunology, Coligan et al., eds., John Wiley & Sons. Epitopes can be confirmed by introducing point mutations or deletions into a known epitope, and then testing binding with one or more antibodies to determine which mutations reduce binding of the antibodies.

Antibodies or antigen-binding fragments of the invention may be used in diagnostic methods alone or in conjunction with certain antibodies already known in the art. Known antibodies may include anti-cytochrome c antibodies as well as anti-acetylation-moiety antibodies, which bind to acetylated polypeptides.

An antibody or antigen-binding fragment thereof of the invention can be linked to a detectable label. A detectable label of the invention may be attached to antibodies or antigen-binding fragments thereof of the invention by standard protocols known in the art. In some embodiments, the detectable labels may be covalently attached to an anti-acetylated cytochrome c antibody or antigen-binding fragment thereof of the invention. The covalent binding can be achieved either by direct condensation of existing side chains or by the incorporation of external bridging moieties. Many bivalent or polyvalent agents are useful in coupling protein molecules to other proteins, polypeptides or amine functions, etc. For example, the literature is replete with coupling agents such as carbodiimides, diisocyanates, glutaraldehyde, and diazobenzenes. This list is not intended to be exhaustive of the various coupling agents known in the art but, rather, is exemplary of the more common coupling agents. Additional descriptions of detectable labels useful in the invention are provided elsewhere herein.

The invention, in part, also includes nucleic acid sequences that encode polypeptide sequences for use in generating antibodies. For example, the invention includes nucleic acid sequences that encode a cytochrome c polypeptide or fragment thereof, and includes the use of the nucleic acid sequences that may be used to produce polypeptides that can be used as antigens with which to raise antibodies that recognize acetylated cytochrome c polypeptides.

Polypeptides and/or nucleic acids of the invention may be detectably labeled for use in methods and/or compositions of the invention. A wide variety of detectable labels are available for use in methods of the invention and may include labels that provide direct detection (e.g., fluorescence, colorimetric, or optical, etc.) or indirect detection (e.g., enzyme-generated luminescence, epitope tag such as the FLAG epitope, enzyme tag such as horseradish peroxidase, labeled antibody, etc.). A variety of methods may be used to detect a detectable label depending on the nature of the label and other assay components. Labels may be directly detected through optical or electron density, radioactive emissions, nonradiative energy transfers, etc. or indirectly detected with antibody conjugates, strepavidin-biotin conjugates, etc. Methods for using and detecting labels are well known to those of ordinary skill in the art. Methods of the invention may be used for in vivo, in vitro, and/or ex vivo imaging, including but not limited to real-time imaging. The presence of a labeled antibody in a subject can be detected by in vivo, ex vivo, or in vitro imaging using standard methods. Examples of detection methods include, but are not limited to, MRI, functional MRI, X-Ray detection, PET, CT imaging, immunohistochemistry, Western blot of tissues or cells, or by any other suitable detection method.

The term “detectable label” as used here means a molecule preferably selected from, but not limited to, fluorescent, enzyme, radioactive, metallic, biotin, chemiluminescent, and bioluminescent molecules. As used herein, a detectable label may be a colorimetric label, e.g., a chromophore molecule. In some aspects of the invention, a polypeptide or an antibody may be detectably labeled with a single or with two or more of the detectable labels set forth herein, or other art-known detectable labels.

Radioactive or isotopic labels may be, for example, ¹⁴C, ³H, ³⁵S, ¹²⁵I, and ³²P. Fluorescent labels may be any compound that emits an electromagnetic radiation, preferably visible light, resulting from the absorption of incident radiation and persisting as long as the stimulating radiation is continued.

Examples of fluorescent labels that may be used on polypeptides and/or antibodies of the invention and in methods of the invention include but are not limited to 2,4-dinitrophenyl, acridine, cascade blue, rhodamine, 4-benzoylphenyl, 7-nitrobenz-2-oxa-1,3-diazole, 4,4-difluoro-4-bora-3a,4a-diaza-3-indacene and fluorescamine. Absorbance-based labels may be molecules that are detectable by the level of absorption of various electromagnetic radiation. Such molecules may be, for example, the fluorescent labels indicated above.

Chemiluminescent labels in this invention refer to compounds that emit light as a result of a non-enzymatic chemical reaction. Methods of the invention may also include the use of a luminescent detectable diagnostic molecule such as enhanced green fluorescent protein (EGFP), luciferase (Luc), or another detectable expression product.

Enzymatic methods for detection may be used including the use of alkaline phosphatase and peroxidase. Additional enzymes may also be used for detection in methods and kits of the invention.

As used herein, fluorophores include, but are not limited to amine-reactive fluorophores that cover the entire visible and near-infrared spectrum. Examples of such fluorophores include, but are not limited to, 4-methylumbelliferyl phosphate, fluorescein isothiocyanate (FITC), tetramethylrhodamine isothiocyanate (TRITC), BODIPY dyes; Oregon Green, rhodamine green dyes; the red-fluorescent Rhodamine Red-X, Texas Red dyes; and the UV light-excitable Cascade Blue, Cascade Yellow, Marina Blue, Pacific Blue and AMCA-X fluorophores. Fluorophores may also include non-fluorescent dyes used in fluorescence resonance energy transfer (FRET).

A labeled polypeptide or antibody of the invention can be prepared from standard moieties known in the art. As is recognized by one of ordinary skill in the art, the labeling process for preparing a detectable labeled polypeptide, antibody, or fragment thereof may vary according to the molecular structure of the polypeptide or antibody and the detectable label. Methods of labeling polypeptides and/or antibodies with one or more types of detectable labels are routinely used and are well understood by those of ordinary skill in the art.

Compositions (e.g., acetylated polypeptides, antibodies to acetylated cytochrome c and derivatives/conjugates thereof, etc.) of the present invention have diagnostic and therapeutic utilities. As detailed herein, the antibodies or antigen-binding fragments thereof of the invention may be used for example to identify and/or isolate cytochrome c polypeptides and/or acetylated and/or non-acetylated cytochrome c polypeptides. The antibodies may be coupled to specific diagnostic labeling agents for imaging of the mutant and/or wild-type cytochrome c polypeptides or fragments thereof. The antibodies or antigen-binding fragments thereof of the invention may also be used for immunoprecipitation, immunoblotting cytochrome c and/or acetylated cytochrome c using standard methods known to those of ordinary skill in the art.

In some embodiments, an antibody or antigen-binding fragment thereof of the invention that specifically binds to an acetylated cytochrome c polypeptide may be in solution or may be attached to a surface (e.g., a dipstick, microtiter plate, multiwell plate, plastic, slide, card, etc.). A sample from a subject may then be applied to the substrate and the substrate is then processed to assess whether specific binding occurs between the antibody and a polypeptide or other component of the sample. As used herein a substrate may be made of a material including any synthetic or natural material. Examples of substrates of the invention may include, but are not limited to: glass, plastic, nylon, metal, paper, cardboard, filter paper, filter membranes, etc., and can be in numerous forms including, but not limited to, tubes, centrifuge tubes, cuvettes, cards, slides, dipsticks, beads, coverslips, multiwell plates, Petri plates, etc. One of ordinary skill in the art will recognize that numerous additional types of surfaces can be used in the methods of the invention.

As will be understood by one of skill in the art, a binding assay using an antibody of the invention may also be performed in solution by contacting a sample from a subject with an antibody or antigen-binding fragment thereof of the invention when the antibody or antigen-binding fragment thereof, for example in a 96-well plate, a tube, a drop on a slide, etc.

As used herein the term “attached to a surface” means chemically or biologically linked to the surface and not freely removable from a surface. Examples of attachment, though not intended to be limiting are covalent binding between the substrate and an antibody, attachment via specific biological binding, or the like. For example, “attached” in this context includes chemical linkages, chemical/biological linkages, etc. As used herein the term “covalently attached” means attached via one or more covalent bonds. As used herein the term “specifically attached” means an antibody or fragment thereof is chemically or biochemically linked to a surface as described above with respect to the definition of “attached,” but excluding all non-specific binding. In the methods of the invention, an antibody that is attached to a substrate is attached such that the antibody is not removable from the substrate without specific stripping methods or solutions. Such stripping methods may include, but are not limited to, physical methods such as scraping or heating, enzymatic methods, and chemical methods, which may include but are not limited to contacting the attached antibody and substrate with a solution such that the link between the substrate and the surface is broken and the substrate is released.

In some embodiments of the invention, an antibody or antigen-binding fragment thereof is attached to a substrate, for example a dipstick, and is contacted with a sample cell or tissue from culture or from a subject. The surface of the substrate may then be processed using procedures well known to those of skill in the art, to assess whether specific binding occurred between the antibody and a polypeptide (e.g., an acetylated cytochrome c polypeptide) in the subject's sample. For example, procedures may include, but are not limited to, contact with a secondary antibody, or other method that indicates the presence of specific binding.

### Administration

The pharmacological agents used in the methods of the invention are preferably sterile and contain an effective amount of one or more agents for producing the desired response in a unit of weight or volume suitable for administration to a subject. The doses of pharmacological agents administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits. The dosage of a pharmacological agent may be adjusted by the individual physician or veterinarian, particularly in the event of any complication. A therapeutically effective amount typically varies from 0.01 mg/kg to about 1000 mg/kg, preferably from about 0.1 mg/kg to about 500 mg/kg, and most preferably from about 0.2 mg/kg to about 250 mg/kg, in one or more dose administrations daily, for one or more days.

Agents associated with the invention and optionally other therapeutics may be administered per se or in the form of a pharmaceutically acceptable salt.

Various modes of administration are known to those of ordinary skill in the art which effectively deliver the pharmacological agents of the invention to a desired tissue, cell, or bodily fluid. The administration methods are discussed elsewhere in the application. The invention is not limited by the particular modes of administration disclosed herein. Standard references in the art (e.g., Remington's Pharmaceutical Sciences, 20th Edition, Lippincott, Williams and Wilkins, Baltimore Md., 2001) provide modes of administration and formulations for delivery of various pharmaceutical preparations and formulations in pharmaceutical carriers. Other protocols which are useful for the administration of pharmacological agents of the invention will be known to one of ordinary skill in the art, in which the dose amount, schedule of administration, sites of administration, mode of administration and the like vary from those presented herein.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A pharmacological agent or composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the pharmacological agents of the invention, and with each other, in a manner such that there is no interaction which would substantially impair the desired pharmaceutical efficacy.

The pharmaceutical compositions may contain suitable buffering agents, as described above, including: acetate, phosphate, citrate, glycine, borate, carbonate, bicarbonate, hydroxide (and other bases) and pharmaceutically acceptable salts of the foregoing compounds. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier, which constitutes one or more accessory ingredients. In general, the compositions are prepared by uniformly and intimately bringing the active compound into association with a liquid carrier, a finely divided solid carrier, or both, and then, if necessary, shaping the product.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle (e.g., saline, buffer, or sterile pyrogen-free water) before use.

Compositions suitable for oral administration may be presented as discrete units, such as capsules, tablets, pills, lozenges, each containing a predetermined amount of the active compound. Other compositions include suspensions in aqueous liquids or non-aqueous liquids such as a syrup, elixir, an emulsion, or a gel.

Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, sorbitol or cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the agent or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, agents may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of an agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery. Agents can be delivered to the lungs of a mammal while inhaling and traverse across the lung epithelial lining to the blood stream. Reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J. Immunol. 140:3482-3488 (interferon-γ and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of a given agent. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise an agent dissolved in water at a concentration of about 0.1 to 25 mg of biologically active agent per mL of solution. The formulation may also include a buffer and a simple sugar (e.g., for stabilization and regulation of osmotic pressure). The nebulizer formulation may also contain a surfactant, to reduce or prevent surface induced aggregation of the agent caused by atomization of the solution in forming the aerosol.

Formulations for use with a metered-dose inhaler device will generally comprise a finely divided powder containing the agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing an agent and may also include a bulking agent, such as lactose, sorbitol, sucrose, or mannitol in amounts which facilitate dispersal of the powder from the device, e.g., 50 to 90% by weight of the formulation. The agent should most advantageously be prepared in particulate form with an average particle size of less than 10 mm (or microns), most preferably 0.5 to 5 mm, for most effective delivery to the distal lung.

Nasal (or intranasal) delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic agent(s), may be provided in particles. Particles as used herein means nano or micro particles (or in some instances larger) which can consist in whole or in part of therapeutic agent(s) described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero order release, first order release, second order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain therapeutic agents described herein in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

For topical administration to the eye, nasal membranes, mucous membranes or to the skin, the therapeutic agents may be formulated as ointments, creams or lotions, or as a transdermal patch or intraocular insert or iontophoresis. For example, ointments and creams can be formulated with an aqueous or oily base alone or together with suitable thickening and/or gelling agents. Lotions can be formulated with an aqueous or oily base and, typically, further include one or more emulsifying agents, stabilizing agents, dispersing agents, suspending agents, thickening agents, or coloring agents. (See, e.g., U.S. Pat. No. 5,563,153, entitled “Sterile Topical Anesthetic Gel”, issued to Mueller, D., et al., for a description of a pharmaceutically acceptable gel-based topical carrier.)

In general, the therapeutic agent is present in a topical formulation in an amount ranging from about 0.01% to about 30.0% by weight, based upon the total weight of the composition. Preferably, the agent is present in an amount ranging from about 0.5 to about 30% by weight and, most preferably, the agent is present in an amount ranging from about 0.5 to about 10% by weight. In one embodiment, the compositions of the invention comprise a gel mixture to maximize contact with the surface of the localized pain and minimize the volume and dosage necessary to alleviate the localized pain. GELFOAM® (a methylcellulose-based gel manufactured by Upjohn Corporation) is a preferred pharmaceutically acceptable topical carrier. Other pharmaceutically acceptable carriers include iontophoresis for transdermal drug delivery.

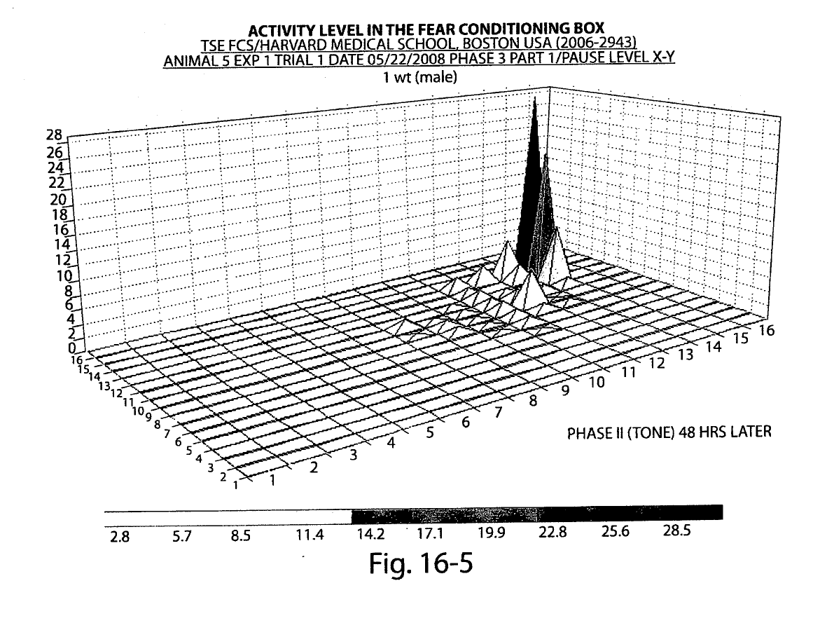

The invention also contemplates the use of kits. In some aspects of the invention, the kit can include a pharmaceutical preparation vial, a pharmaceutical preparation diluent vial, and one or more therapeutic agents. In some embodiments the kit contains agents for diagnostic purposes such as an antibody or multiple antibodies. The vial containing the diluent for the pharmaceutical preparation is optional. The diluent vial contains a diluent such as physiological saline for diluting what could be a concentrated solution or lyophilized powder of a therapeutic agent. The instructions can include instructions for mixing a particular amount of the diluent with a particular amount of the concentrated pharmaceutical preparation, whereby a final formulation for injection or infusion is prepared. The instructions may include instructions for treating a subject with an effective amount of a therapeutic agent. The instructions may include instructions for diagnosing a patient, characterizing a patient's risk for a given disease, or evaluating the effectiveness of a given therapy for a patient. It also will be understood that the containers containing the preparations, whether the container is a bottle, a vial with a septum, an ampoule with a septum, an infusion bag, and the like, can contain indicia such as conventional markings which change color when the preparation has been autoclaved or otherwise sterilized. A kit associated with the invention is presented in FIG. 17.

The present invention is further illustrated by the following Example, which in no way should be construed as further limiting. The entire contents of all of the references (including literature references, issued patents, published patent applications, and co-pending patent applications) cited throughout this application are hereby expressly incorporated by reference.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### Examples

### Methods

### Cell Culture and Transfection

SH-SY5Y cells were cultured in Dulbecco's Modified Eagle's Medium (DMEM) supplemented with 10% Fetal Calf Serum (FCS). SH-SY5Y cells were transfected with Lipofectamine 2000 (Invitrogen, Carlsbad, Calif.). Granule neuron cultures were prepared from cerebella of postnatal day 5 mouse pups. Neurons were placed on polyornithine-coated 96-well plates and grown in Basal Medium Eagle (BME) (Sigma, St. Louis, Mo.) supplemented with 10% calf serum (Hyclone Laboratories, Logan, Utah), 25 mM KCl, 2 mM glutamine, penicillin, and streptomycin. One day after cultures were prepared they were treated with 10 μM of the antimitotic agent cytosine-D-arabinofuranoside (Sigma, St. Louis, Mo.) to prevent the proliferation of non-neuronal cells.

### Plasmid Construction

All expression constructs were generated by using PCR-based standard cloning strategies, and all expression constructs were verified by DNA sequencing. Mouse cytochrome c coding sequence was PCR amplified from pEGFP-mouse Cytochrome c-GFP vector (a gift from D. Green, St. Jude Children's Research Hospital, Memphis, Tenn.) and cloned into the pcDNA3.1+ (Invitrogen)-derived vector pcDNAFlag to yield cytochrome c with a C-terminal Flag tag. Site-directed mutagenesis was used to construct pcDNA-mouse cytochrome c K74R-Flag, pcDNA-mouse cytochrome c K40R-Flag and pcDNA-mouse cytochrome c K88R-Flag. All constructs were verified by DNA sequencing. pcDNA-human SIRT3-Flag and pcDNA-human SIRT3-HA vector were provided by B. Schwer, University of California, San Francisco.

### Immunoblotting

Antibodies used were anti-Flag M2, rabbit polyclonal anti-Flag and anti-FLAG M2 agarose affinity gel (Sigma, St. Louis, Mo.), anti-HA monoclonal antibodies (Sigma), acetylated-lysine polyclonal antibody (Cell Signaling Technology, Beverly, Mass.), anti-cytochrome c (Pharmingen and Santa Cruz Biotechnology). Immunoblots were developed with enhanced chemiluminescence (GE Healthcare).

### Immunoprecipitation

Cells were lysed in ice-cold buffer IP buffer (1% Triton X-100, 150 mM NaCl, 0.5 mM EDTA, 50 mM Tris-HCl, pH 7.4) containing protease inhibitor cocktail (Roche). Lysates were centrifuged at 16,000 g for 10 min at 4° C., and immunoprecipitation was performed at 4° C. for 12 h by using anti-FLAG M2 agarose affinity gel (Sigma, St. Louis, Mo.). When anti-cytochrome c (Santa Cruz Biotechnology) antibody was used for immunoprecipitation, the samples were incubated for 4 h at 4° C. Samples were washed four times in IP buffer. Purified proteins were separated by SDS-PAGE and immunoblotting was performed.

### Large-Scale Purification of Cytochrome c Followed by Tandem Mass Spectrometry

A large-scale Flag-immunoprecipitation followed by elution with 3× Flag peptide was performed on cell extracts from 10×15 cm-plates of SH-SY5Y cells transfected with pcDNA-mouse cytochrome c-Flag+/−pcDNA-hSIRT3-HA. Purified proteins were separated by SDS-PAGE, and the band corresponding to cytochrome c was excised and analyzed by MS/MS.

### Cell Survival Measurements

The resistance to kainic acid of primary granule neurons derived from WT and SIRT3 null mice was quantified as the percentage of live cells after a 4 hr and 24 hrs treatment with 50 uM Kainic acid. The quantitation of dead cells upon Kainic acid treatment was carried out using the MTT assay was performed using CellTiter 96 Nonradioactive Cell Proliferation Assay kit (Promega).

### Animal Experimentation

Littermate 129/Sv (6-8 weeks old) mice were used for the studies. SIRT3−/− mice were in a 129/sv background. Mice were housed under controlled temperature (25° C.) and light, and fed normal chow.

To induce seizures, both WT female (n=6) and KO female (n=5) mice and WT male (n=5) and KO male (n=6) mice were injected i.p. with 30 mg/kg KA. This dose of KA caused seizures in all mice.

The fear-conditioning experiments were performed using a computerized fear-conditioning system (TSE, Bad Homburg, Germany). Fear conditioning was performed in a cage (36 cm×21 cm×20 cm). The box was cleaned after each trial with 95% ethanol.

### Context-Dependent Fear Conditioning

Training consisted of a 3 min exposure of mice to the conditioning box (context) followed by a foot shock (2 sec, 0.7 mA, constant current). The memory test was performed 24 hr later by re-exposing the mice for 3 min into the conditioning context. Freezing, defined as a lack of movement except for heart rate and respiration associated with a crouching posture, was recorded every 10 sec by two trained observers (one was unaware of the experimental conditions) during 3 min (a total of 18 sampling intervals). The number of observations indicating freezing obtained as a mean from both observers was expressed as a percentage of the total number of observations. Control groups of mice were exposed to the context alone (3 min) or immediate foot shock (2 sec, 0.7 mA, constant current) followed by context (3 min) during the training.

### Tone-Dependent Fear Conditioning

Training consisted of a 3 min exposure of mice to the conditioning box (context), followed by a tone [30 sec, 10 kHz, 75 dB sound pressure level (SPL)] and a foot shock (2 sec, 0.7 mA, constant current). The memory test was performed 24 hr later by exposing the mice for 3 min into a novel context followed by an additional 3 min exposure to a tone (10 kHz, 75 dB SPL). Freezing was recorded every 10 sec by two nonbiased observers as described above.

### Rotarod

After mice became familiarized with the procedure, they were placed on the rotarod (TSE, Bad Homburg, Germany), and the time until the kainic injected mouse would fall off the rotating rod at a novel program to the mouse (18 rpm) was measured. Six trials were performed.

### Open Field Test

Mice were placed into the center of the open-field apparatus (44×44×30 cm). Movements of the animals were tracked by an automatic monitoring system (TSE Systems, Bad Homburg, Germany) for 10 min.

### Water Maze Test

The water maze paradigm was performed in a circular tank filled with opaque water. A platform (11×11 cm) was submerged below the water's surface in the center of the target quadrant. The swimming path of the mice was recorded by a video camera and analyzed by the Videomot 2 software (TSE). For each training session, the mice were placed into the maze subsequently from four random points of the tank. Mice were allowed to search for the platform for 60 s. If the mice did not find the platform within 60 s, they were gently guided to it. Mice were allowed to remain on the platform for 30 s. During the memory test (probe test), the platform was removed from the tank, and the mice were allowed to swim in the maze for 60 s.

### Results

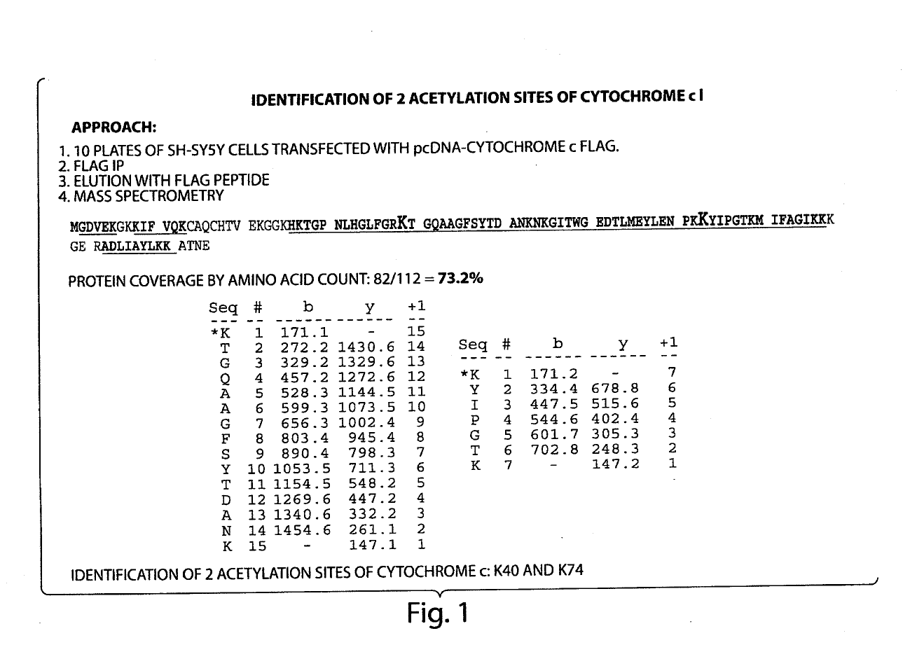

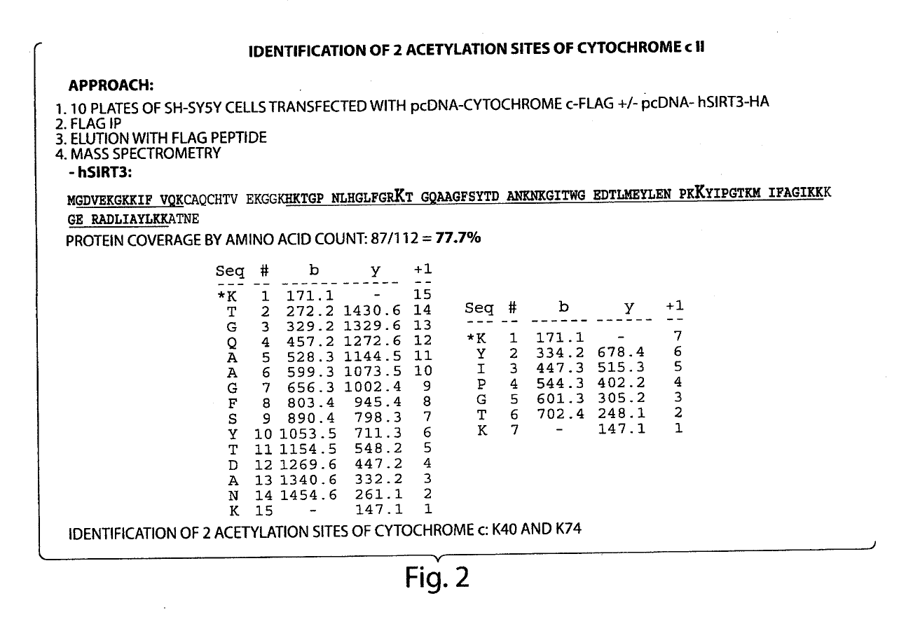

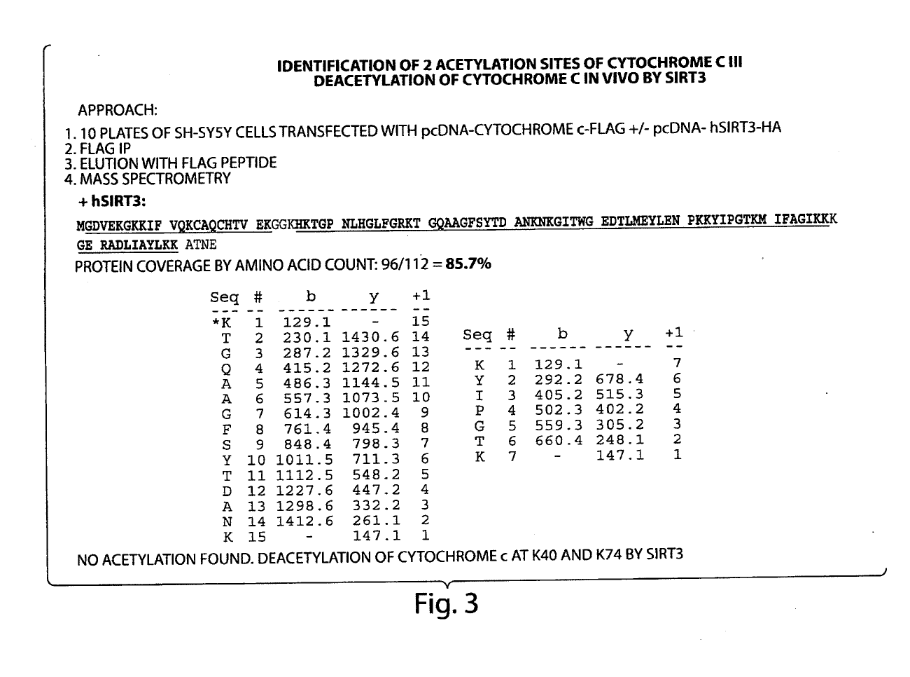

Mass spectrometry analysis revealed that residues K40 and K74 of cytochrome c protein were acetylated in SH-SY5Y cells (FIG. 1). When SH-SY5Y cells were transfected with the cytochrome c protein in the absence of hSIRT3, acetylation of residues K40 and K74 of cytochrome c was detected (FIG. 2), whereas when hSIRT3 was cotransfected with cytochrome c, acetylation of residues K40 and K74 of cytochrome c was no longer detected (FIG. 3), indicating that SIRT3 deacetylates cytochrome c protein. Cytochrome c protein is conserved in a variety of species (FIG. 4).

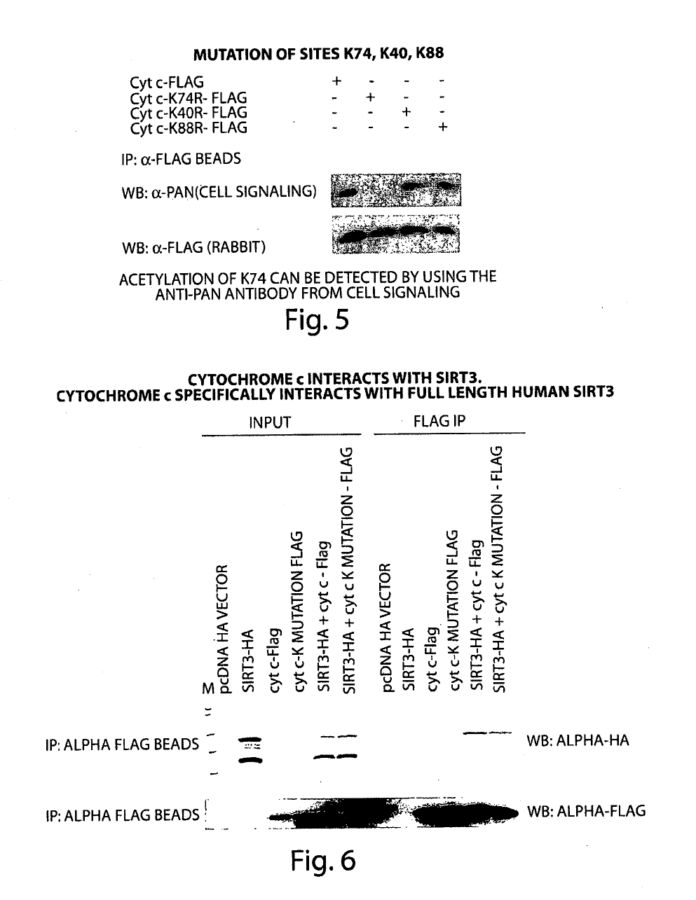

Residues K40, K73 and K88 were mutated by substituting each residue with an arginine residue. Immunoprecipitation experiments revealed that acetylation of K74 can be detected using the anti-PAN antibody (acetylated-lysine polyclonal antibody) (Cell Signaling Technology, Beverly, Mass.) (FIG. 5).

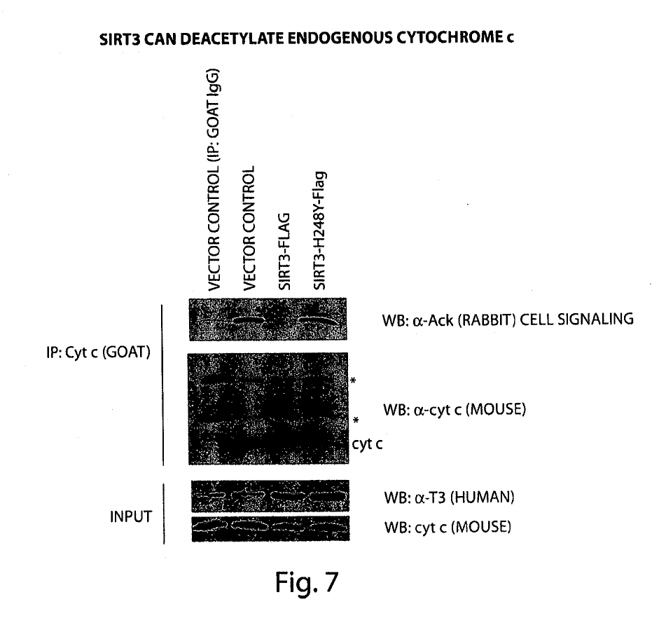

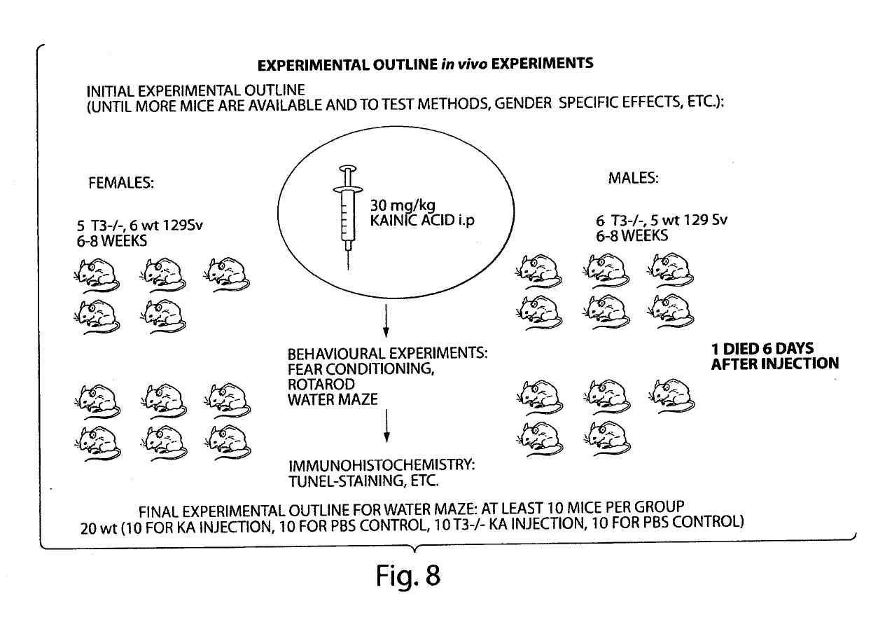

In order to investigate whether SIRT3 interacts with and deacetylates cytochrome c, coimmunoprecipitation experiments and deacetylation experiments were performed. FIGS. 6 and 7 demonstrate an interaction between SIRT3 and cytochrome c, and deacetylation of cytochrome c by SIRT3.

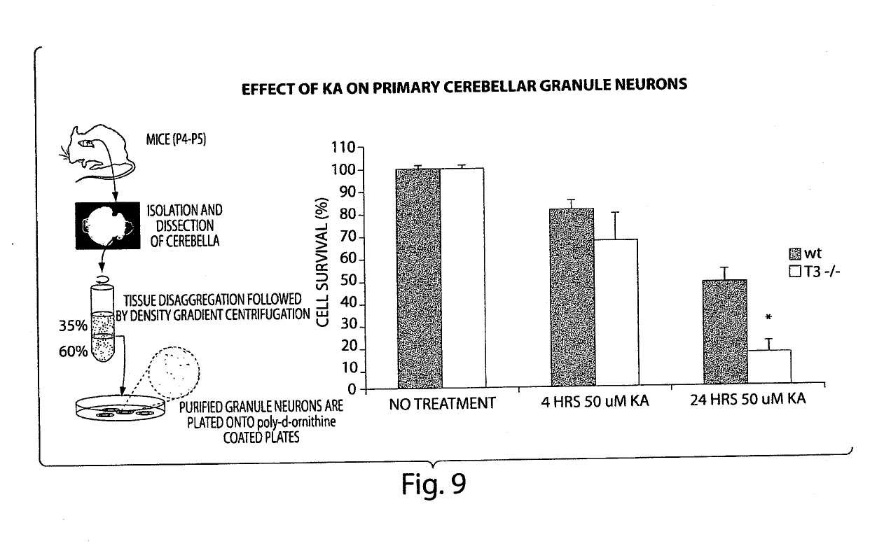

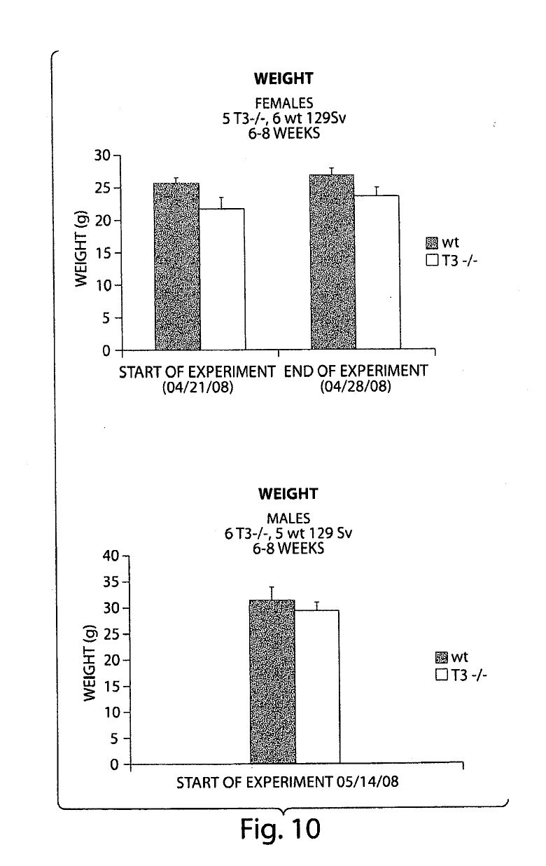

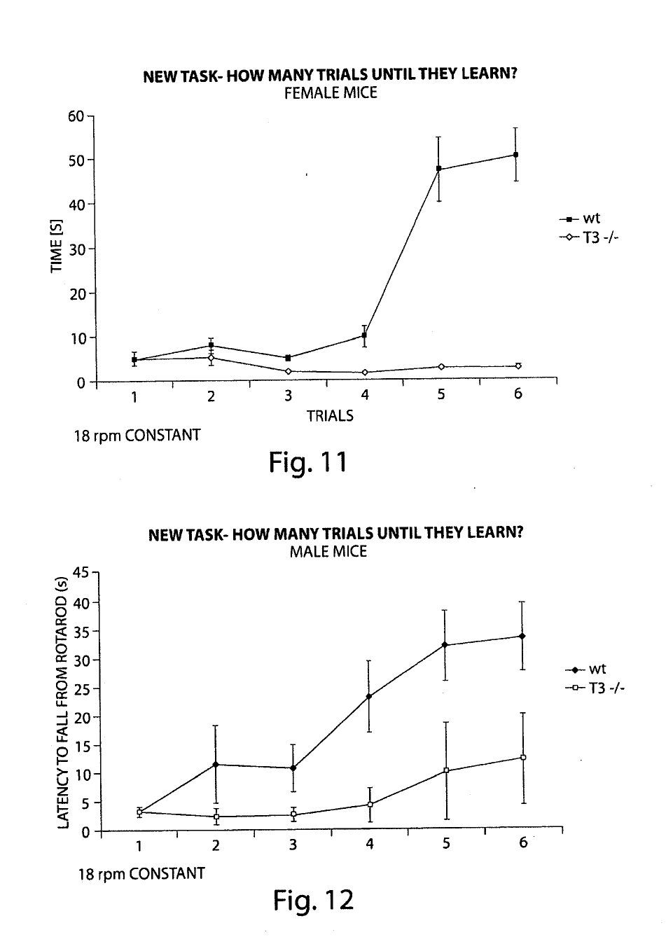

In order to investigate the function of SIRT3, SIRT3 knockout mice were generated (T3−/−). FIG. 8 presents an outline of experimental procedures conducted using T3−/− mice. FIG. 9 reveals the effect of kainic acid on primary cerebellar granule neurons. T3−/− mice exhibit reduced cell survival following kainic acid injection. FIG. 10 reveals a comparison of weights between wild-type and T3−/− mice during the experiments involving kainic acid injection.

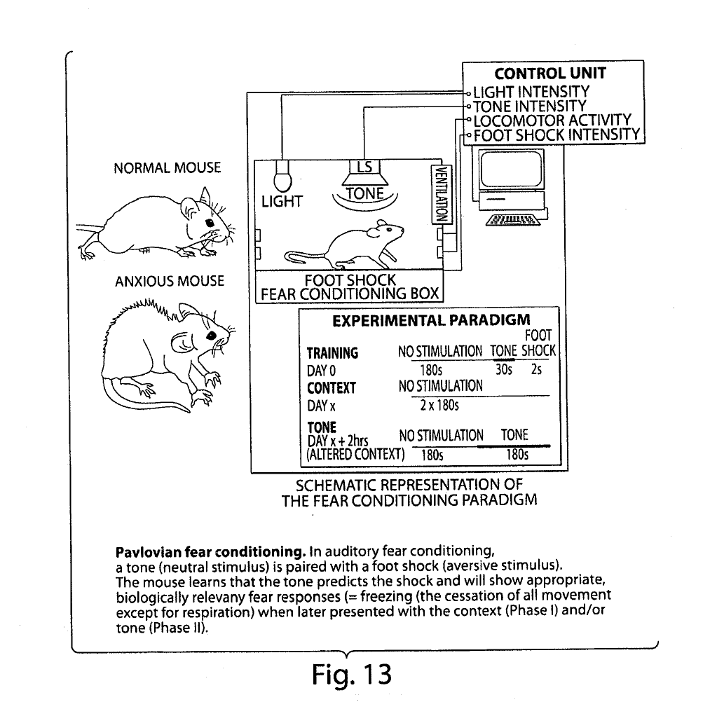

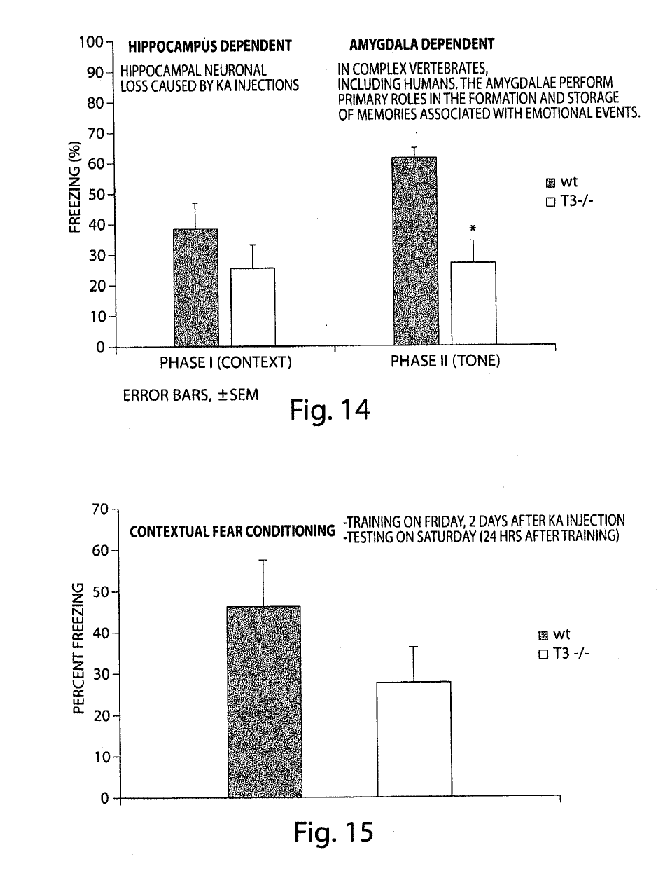

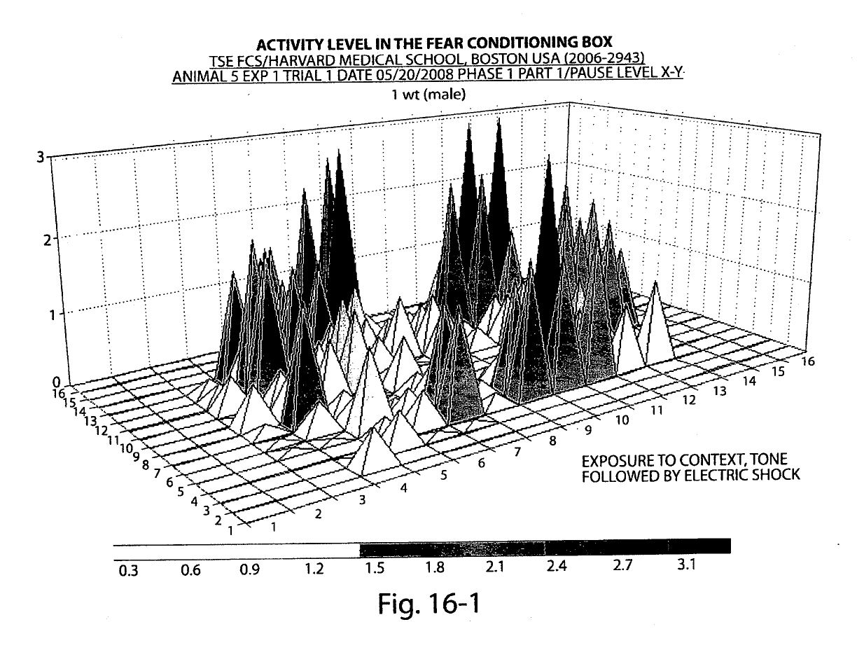

Mice were subjected to fear conditioning experiments and the learning time was compared between wild-type and T3−/− mice. FIGS. 11 and 12 reveal the effect of the T3−/− mutation on learning time. A schematic demonstrating fear conditioning experiments is presented in FIG. 13.

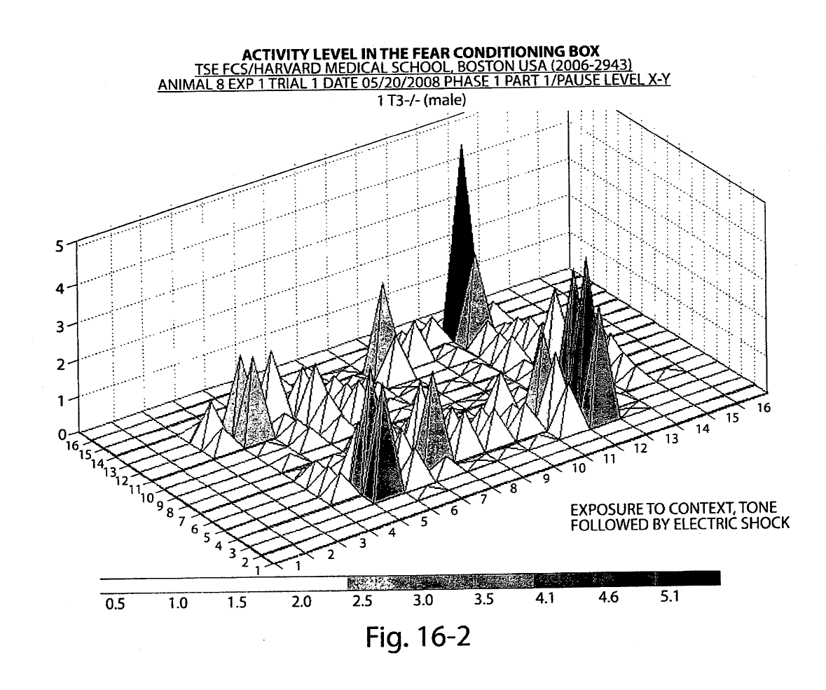

The effect of kainic acid injections on memory function was also investigated by examining hippocampus and amygdala neurons. FIG. 14 demonstrates a loss of hippocampus and amygdala neurons in T3−/− mice.

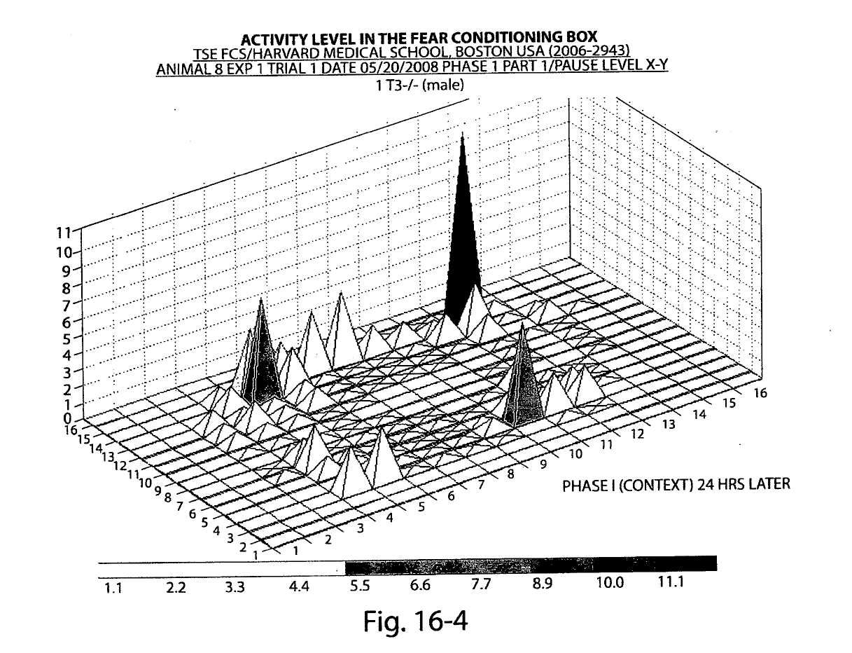

Contextual fear conditioning experiments revealed that T3−/− mice exhibited a lower percentage of freezing activity than wild-type mice that were subjected to fear conditioning. Graphs revealing activity levels in fear conditioning experiments are presented in FIG. 16.

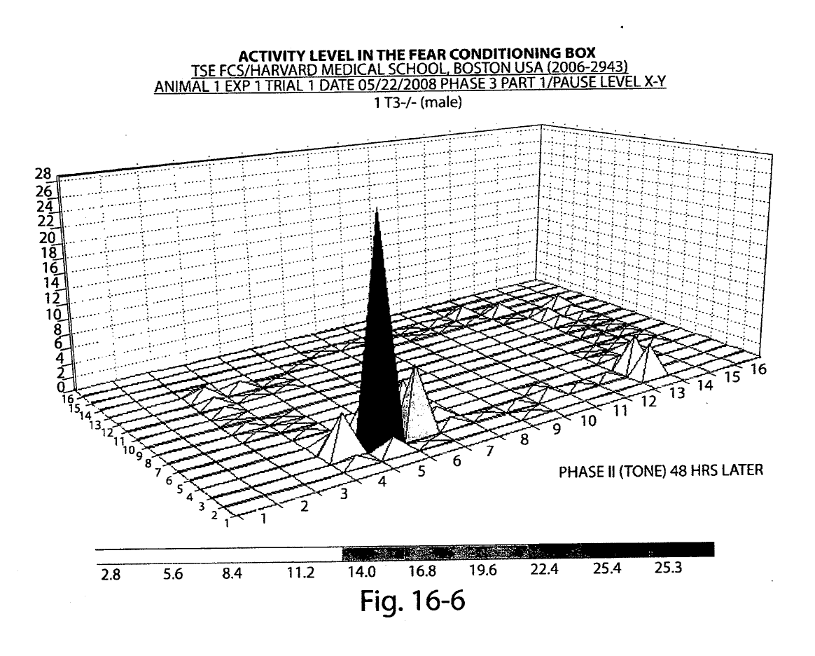

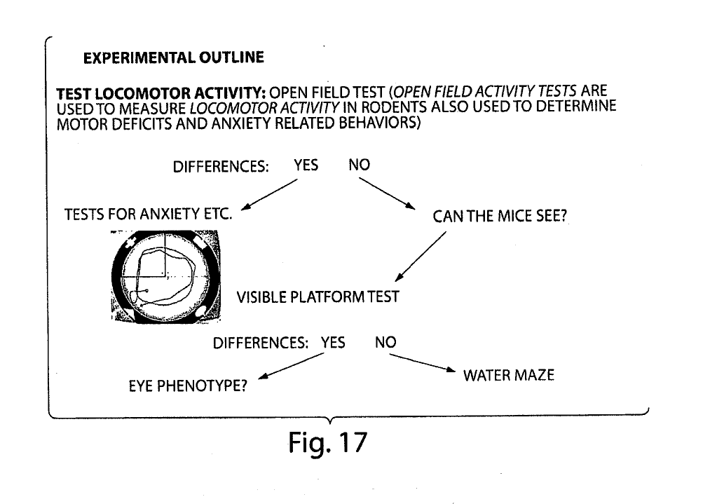

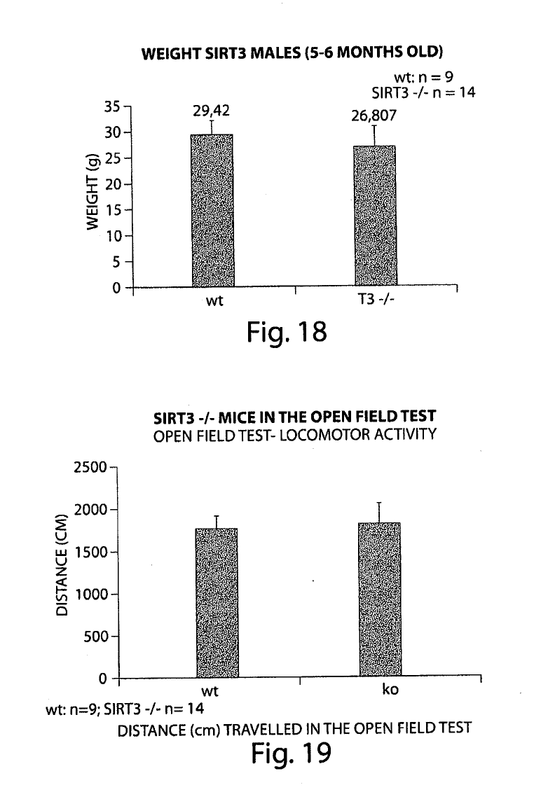

In order to investigate the role of SIRT3 in behavior, T3−/− mice were subjected to behavioral analysis including the Open Field test to test for locomotor activity. A flow-chart indicating the experimental procedures used for evaluating locomotor activity is presented in FIG. 17. The weights of T3−/− and wild-type mice used in locomotor activity experiments are depicted in FIG. 18. The distance traveled and the speed of movement by T3−/− and wild-type mice were tested. T3−/− mice were found to travel a slightly greater distance and to move at a slightly faster speed in the Open Field test than wild-type mice (FIGS. 19-20).

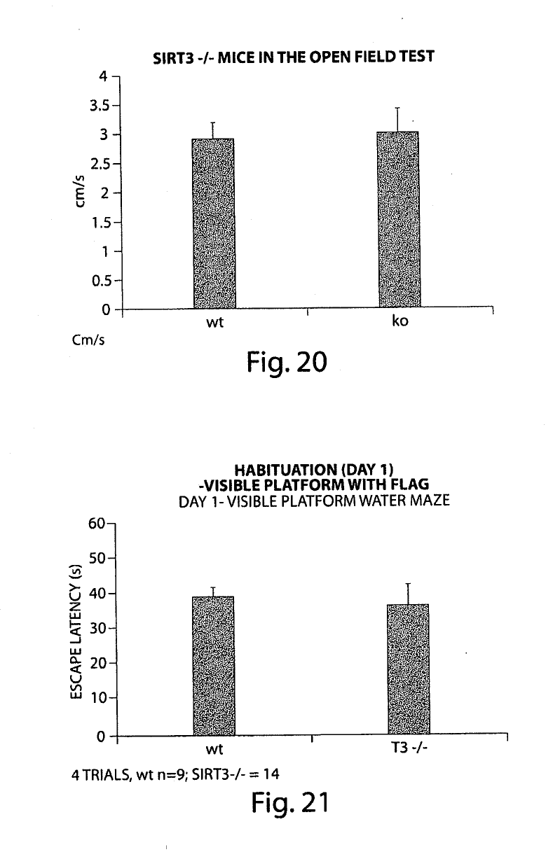

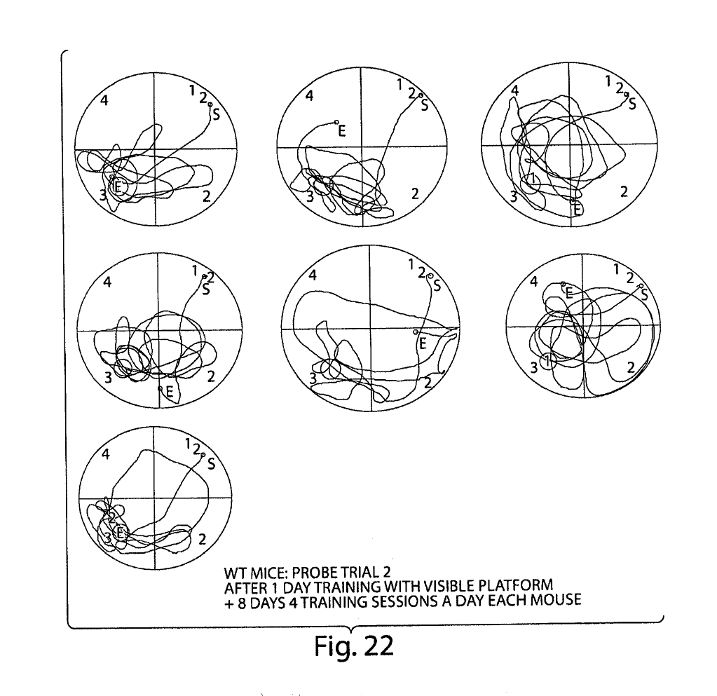

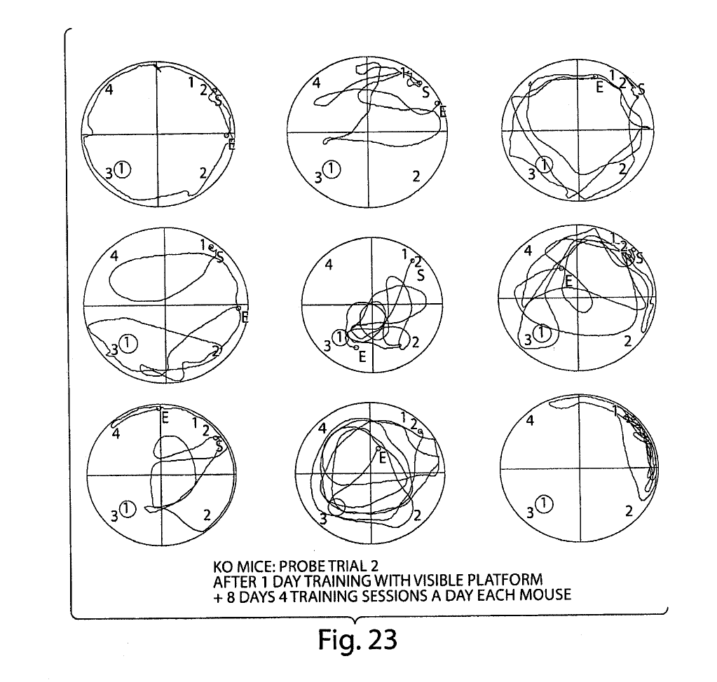

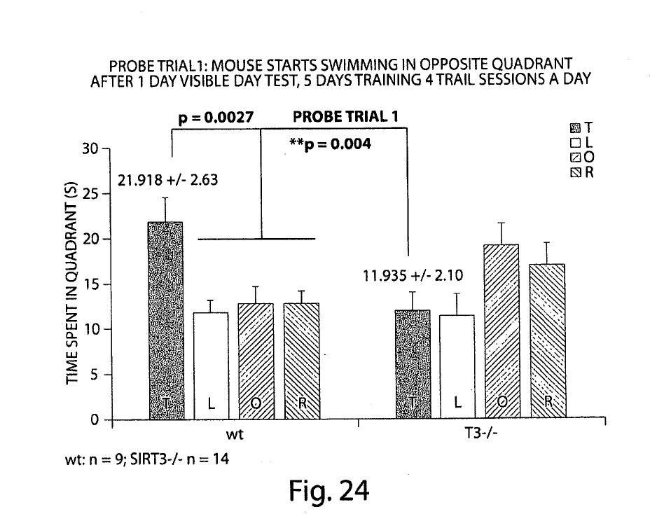

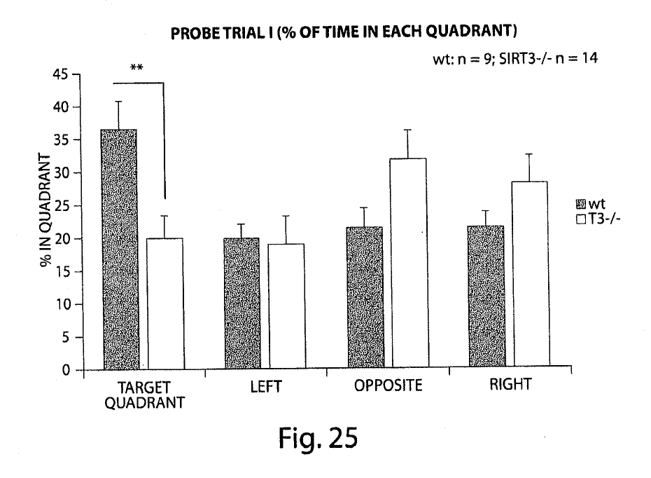

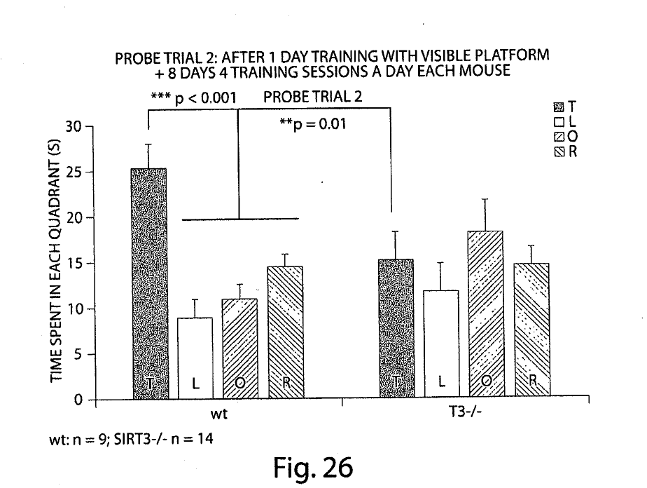

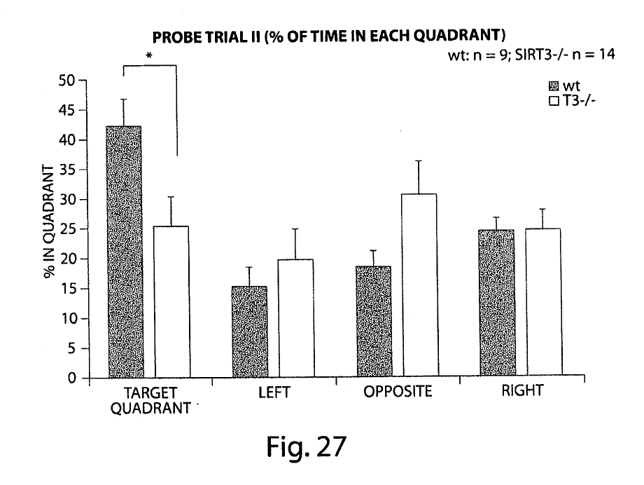

Mice were then tested for locomotor ability in the Water Maze test. During the habituation stage on day 1, T3−/− mice were found to be slightly less able than wild-type mice to escape from the water maze (FIG. 21). After multiple days of training, the movement of mice in the water maze was tracked (FIGS. 22-23). In Trial 1, T3−/− mice were found to spend less time in the target quadrant than wild-type mice, and more time in the opposite quadrant than wild-type mice (FIGS. 24-25). The same results were observed in Trial 2 (FIGS. 26-27). These results indicate a reduced learning capacity for T3−/− mice in locomotor activity tests relative to wild-type mice.

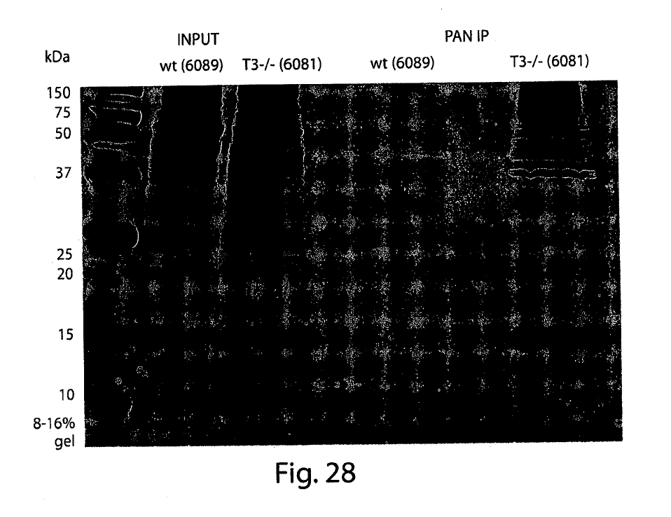

In order to determine the acetylation status of proteins in the hippocampus of T3−/− mice, immunoprecipitation experiments were conducted from hippocampal lysates from wild-type and T3−/− mice using the PAN antibody (Cell Signaling Technology, Beverly, Mass.). These experiments revealed hyperacetylation of proteins in the hippocampus of T3−/− mice (FIG. 28).

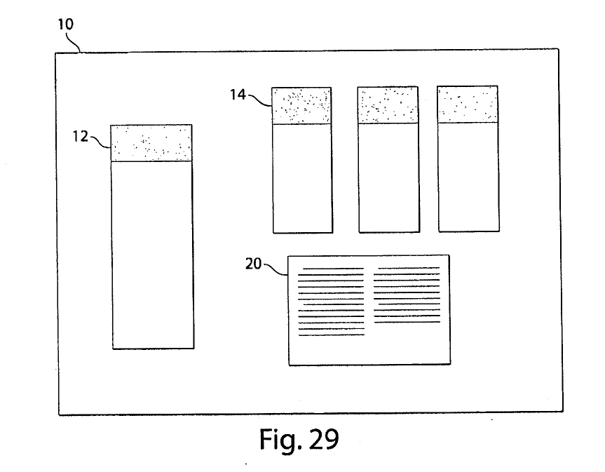

A schematic of a kit associated with the invention is presented in FIG. 29.

### EQUIVALENTS

Those skilled in the art will recognize, or be able to ascertain using no more than routine experimentation, many equivalents to the specific embodiments of the invention described herein. Such equivalents are intended to be encompassed by the following claims. All references, including patent documents, disclosed herein are incorporated by reference in their entirety.

What is claimed is:

1.-9. (canceled)

10. A method for diagnosing a neurodegenerative disorder in a subject, the method comprising:
detecting the level of acetylated cytochrome c in a sample from the subject,
wherein an elevated level of acetylated cytochrome c in the sample from the subject, relative to a predetermined value, is indicative of a neurodegenerative disorder.

11. The method of claim 10 wherein the level of acetylated cytochrome c in a sample from the subject is detected by using an antibody that specifically binds acetylated cytochrome c.

12. The method of claim 10 wherein the level of acetylated cytochrome c in a sample from the subject is detected by mass spectrometry.

13. A method for evaluating the efficacy of a therapy in a subject with a neurodegenerative disorder, the method comprising:
detecting the level of acetylation of cytochrome c in a sample from the subject,
wherein the level of acetylation of cytochrome c in the sample from the subject, relative to a predetermined value, is indicative of whether the therapy is efficacious.

14. The method of claim 13 wherein the level of acetylated cytochrome c in a sample from the subject is detected by using an antibody that specifically binds acetylated cytochrome c.

15. The method of claim 13 wherein the level of acetylated cytochrome c in a sample from the subject is detected by mass spectrometry.

16.-45. (canceled)

46. A method for identifying a compound that modulates the deacetylase activity of SIRT3, comprising:
contacting an acetylated cytochrome c polypeptide substrate and a SIRT3 deacetylase in the presence of a test compound, and
determining the level of acetylation of the cytochrome c polypeptide substrate in the presence of the test compound, wherein a decrease in the level of acetylation of the cytochrome c polypeptide substrate in the presence of the test compound as compared to a control is indicative of a compound that increases SIRT3 deacetylase activity, and wherein an increase in the level of acetylation of the cytochrome c polypeptide substrate in the presence of the test compound as compared to a control is indicative of a compound that decreases SIRT3 deacetylase activity.

47. The method of claim 46, wherein the cytochrome c polypeptide substrate comprises at least one acetylated lysine residue corresponding to residues K40 and/or K74 of full-length, wild-type human cytochrome c polypeptide.

48. The method of claim 46, wherein the level of acetylation of the cytochrome c polypeptide substrate pool is determined using mass spectrometry.

49. The method of claim 48, wherein the mass spectrometry is electrospray ionization (ESI) mass spectrometry or matrix-assisted laser desorption/ionization (MALDI) mass spectrometry.

50. The method of claim 46, wherein the cytochrome c polypeptide substrate comprises a single polypeptide species.

51. The method of claim 46, wherein the cytochrome c polypeptide substrate comprises a full-length cytochrome c polypeptide.

52. The method of claim 46, wherein the cytochrome c polypeptide substrate comprises a mixture of two or more polypeptides species.

53. The method of claim 46, wherein the cytochrome c polypeptide substrate is a fragment of cytochrome c comprising at least one lysine residue corresponding to residues K40 and/or K74 of full-length, wild-type human cytochrome c polypeptide.

54. The method of claim 46, wherein the cytochrome c polypeptide substrate is a fusion of a fragment of cytochrome c comprising at least one lysine residue corresponding to residues K40 and/or K74 of full-length, wild-type human cytochrome c polypeptide.

55. The method of claim 46, wherein the test compound is a small molecule.

56. The method of claim 46, wherein the test compound is a library of molecules.

57. The method of claim 56, wherein the library comprises small molecules.

58. The method of claim 46, wherein the SIRT3 deacetylase is from a cell or tissue lysate.

59. The method of claim 46, wherein the cytochrome c polypeptide substrate is in a cell.

60. The method of claim 46, wherein the SIRT3 is a catalytically active fragment of full-length (human) SIRT3 capable of deacetylating a cytochrome c substrate comprising acetylated K40 and/or K74 in the presence of NAD⁺ or a NAD⁺ analog.

61.-63. (canceled)


SEQUENCE LISTING (4 sequences)

SEQ ID NO: 1 - protein, 105 aa, Mus musculus
MGDVEKGKKIFVQKCAQCHTVEKGGKHKTGPNLHGLFGRKTGQAAGFSYTDANKNKGITWGEDTLMEYLENPKKYIPGTKMIFAGIKKKGERADLIAYLKKATNE

SEQ ID NO: 2 - protein, 105 aa, Homo sapiens
MGDVEKGKKIFIMKCSQCHTVEKGGKHKTGPNLHGLFGRKTGQAPGYSYTAANKNKGIIWGEDTLMEYLENPKKYIPGTKMIFVGIKKKEERADLIAYLKKATNE

SEQ ID NO: 3 - protein, 108 aa, Drosophila melanogaster
MGVPAGDVEKGKKLFVQRCAQCHTVEAGGKHKVGPNLHGLIGRKTGQAAGFAYTDANKAKGITWNEDTLFEYLENPKKYIPGTKMIFAGLKKPNERGDLIAYLKSATK

SEQ ID NO: 4 - protein, 109 aa, Saccharomyces cerevisiae
MTEFKAGSAKKGATLFKTRCLQCHTVEKGGPHKVGPNLHGIFGRHSGQAEGYSYTDANIKKNVLWDENNMSEYLTNPKKYIPGTKMAFGGLKKEKDRNDLITYLKKACE